(19) United States
(12) Patent Application Publication
(10) Pub. No.: US 2019/0111111 A1
(43) Pub. Date: Apr. 18, 2019

(54) Treatment of Cerebral Cavernous Malformations

(71) Applicant: The Regents of the University of California, Oakland, CA (US)

(72) Inventors: Mark H. Ginsberg, San Diego, CA (US); Miguel Alejandro Lopez-Ramirez, San Diego, CA (US)

(21) Appl. No.: 16/090,619

(22) PCT Filed: Apr. 13, 2017
(86) PCT No.: PCT/US2017/027368

§ 371 Date: Oct. 2, 2018

Related U.S. Application Data
(60) Provisional application No. 62/321,860, filed on Apr. 13, 2016.

(51) Int. Cl. A61K 38/39; A61P 9/14; C07K 14/78; C07K 14/705

(52) U.S. Cl. CPC A61K 38/39; A61P 9/14; A61K 38/005; C07K 14/70596; C07K 14/78

(57) ABSTRACT

Pharmaceutical compositions, and methods of use thereof, for treating cerebral cavernous malformations and symptoms associated therewith. The pharmaceutical compositions include a therapeutically effective amount of a thrombospondin 1 protein agent. A thrombospondin 1 protein agent can include thrombospondin 1 protein, a functional fragment of thrombospondin 1 protein, an isomer, a homolog, or a peptidomimetic of thrombospondin 1 protein or a functional fragment thereof. The pharmaceutical compositions and methods can further comprise a Rho Kinase inhibitor.

### CROSS-REFERENCE TO RELATED APPLICATIONS

This application claims priority to U.S. Provisional Patent Application Ser. No. 62/321,860 filed on Apr. 13, 2016, the entire contents of which are hereby incorporated.

### STATEMENT REGARDING FEDERALLY SPONSORED RESEARCH

This invention was made with government support under Grant Nos. NS092521 and HL106489 awarded by the National Institutes of Health. The government has certain rights in the invention.

### FIELD OF THE INVENTION

The present disclosure relates generally to the treatment of cerebral cavernous malformations.

### BACKGROUND OF THE INVENTION

Cerebral cavernous malformations (CCM) is a neurovascular disease that causes epilepsy and stroke and for which there is no medical therapy. It has a prevalence of 5 per thousand in western populations and occurs in familial forms as a consequence of mutations in 3 genes: KRIT1, CCM2, PCDC10. Once identified, CCM patients have a lifetime risk of CCM development and progression with resulting risk of stroke, epilepsy, and neurological impairment.

Cerebral cavernous malformations (CCMs) are central nervous system vascular anomalies that lead to significant morbidity and mortality¹. CCMs affect ˜1/200 humans and cause a lifelong risk of stroke and other neurological sequelae for which there is no pharmacologic therapy. Loss of function mutations of three genes (KRTI1, CCM2, PDCD10) are associated with development of venous capillary dysplasia's with hemorrhage and increased vascular permeability² characteristic of CCM^{3,4}. The KRIT1^{+/-} genotype is the most common cause of the familial form of CCM⁵. In mice, timed endothelial-specific inactivation of Krit1 results in cerebellar and retinal vascular lesions that are similar to those in CCM patients⁶⁻⁸. These murine studies, in combination with the finding of a “second hit” on the normal KRIT1 allele in CCM endothelial cells⁹ indicate that a complete loss of KRIT1 function causes endothelial cell autonomous CCM formation.

The consequences of loss of endothelial KRIT1 include abnormal angiogenesis^{6,10}, dysregulation of endothelial metalloproteinases, increased expression of the transcription factors KLF2 and KLF4^{7,11,12} and alterations in signaling pathways such as Notch¹³, VEGF¹⁴ and Rho/ROCK^{15,16}. In addition, increased cell migration due to disruption of endothelial apical-basal polarity¹⁷ and endothelial-mesenchymal transition⁷ have been recently reported to be features of CCMs. Changes in gene expression associated with these phenotypic changes, including increased expression of the transcription factors KLF2 and KLF4^{11,12} and mesenchymal genes^{7,8}; however, a detailed picture of the early changes in gene expression that follow loss of KRIT1 has been lacking.

### SUMMARY OF THE INVENTION

Provided are methods and pharmaceutical compositions for treatment and prevention of cerebral cavernous malformations (CCM). The methods of the invention include administering to a subject in need a thrombospondin 1 (TSP) agent. Exemplary TSP agents include TSP protein, or a biologically active fragment thereof, or a protein mimetic thereof. Recombinant biologically active fragments TSP protein, such as 3TSR, are demonstrated to be therapeutic in an exemplary mouse model of the CCM disease. Peptide mimetics of this recombinant protein, such as ABT-510 are also examples of TSP agents. TSP agent therapies are provided and small molecule orally-available TSP agents can be used to treat CCM patients.

Previous studies identified a role for RhoA/Rho Kinase in the pathogenesis of CCM disease and showed the efficacy of Rho Kinase (ROCK) inhibitors. Studies show that inhibiting ROCK does not prevent the loss of TSP, thus this new approach affects a distinct pathway from ROCK inhibitors and can replace or complement therapy with ROCK inhibitors.

In embodiments, the invention provides methods of treatment and compositions that are homeopathic in the sense that they replace a function of the TSP protein that is lost as a consequence of the pathogenesis of CCM disease.

In embodiments, the invention provides active fragments of TSP or peptides or small molecules that mimic TSP administered to patients with CCM to prevent or treat lesion development and progression and to revert lesions. In embodiments, the invention provides that administration of 3TSR (a biologically active fragment of TSP) or ABT-510 (a peptide mimic of 3TSR) prevent lesion formation in CCM.

In embodiments, the invention provides for manufacture of a medicament for treating or preventing cerebral cavernous malformations, comprising manufacturing a pharmaceutical composition comprising a therapeutically effective amount of a thrombospondin 1 protein agent to treat or prevent cerebral cavernous malformations in the patient.

In embodiments, the invention provides a pharmaceutical composition comprising a therapeutically effective amount of a thrombospondin 1 protein agent and optionally in combination with a Rho Kinase inhibitor to treat or prevent cerebral cavernous malformations in a patient.

### BRIEF DESCRIPTION OF THE DRAWINGS

FIGS. 1a-1h. Loss of KRIT1 inhibits the expression of TSP1. FIG. 1a, Genome wide RNAseq from three independent biological replicates follow by gene ontology analysis of genes differentially expressed in Krit1^{ECKO} BMEC compared to Krit1^{fl/fl} BMEC. Each term listed was the top term in a cluster of related terms and the corrected P values were calculated according to Benjamini's method⁴⁵. FIG. 1b, The expression levels of differentially expressed genes represented on a scatter plot, Fragments Per Kilobase of transcript per Million mapped reads (FPKM) of individual transcripts are represented on a log 2 scale. A few of the most highly suppressed and upregulated genes are labeled. FIG. 1c, RT-qPCR confirmation of RNAseq-identified marked decrease in mRNA of extracellular regulators of angiogenesis in Krit1^{ECKO} BMEC compared to Krit1^{fl/fl} BMEC (S.E.M., N=3). FIG. 1d, Quantification of TSP1 protein from three independent biological replicates in Krit1^{ECKO}(KO) and in Krit1^{fl/fl} (Flox) BMEC (S.E.M., N=3). FIG. 1e, RT-qPCR analysis of isolated brain microvasculature in Krit1^{ECKO} compared to Krit1^{fl/fl} littermate controls (S.E.M., N=3). FIG. 1f, Quantification of TSP1 protein from freshly isolated brain microvasculature in Krit1^{ECKO} (KO) compared to Krit1^{fl/fl} (Flox) littermate controls (S.E.M., N=3). FIG. 1g, Confocal microscopy of cerebellar cortex stained for TSP1 (red) and an endothelial specific marker PECAM1 (green), DAPI staining (blue) was used to reveal nuclei (N=3). FIG. 1h, Higher magnification images of boxed areas in FIG. 1g. TSP1 protein expression was decreased in CCM from Krit1^{ECKO} mice (arrow). Histological analysis of the same region, four sections from the section stained, is showed in FIGS. 8a-8c. Scale bars, 100 m FIG. 1g, 25 μm FIG. 1h. *P<0.05, **P<0.01, ***P<0.001.

FIGS. 2a-2h. Altered tight junctions are an early phenotypic consequence of Krit1 inactivation. FIG. 2a, Representative confocal images of ZO1 (red), claudin5 (CLDN5) (turquoise) and VE-cadherin (green) staining in primary BMEC Krit1^{ECKO} or control Krit1^{fl/fl} BMEC. Nuclei were counterstained with DAPI (blue) (N=4). FIG. 2b, Quantification of brain endothelial ZO1, claudin5 and VE-cadherin protein as assessed by Western Blot analysis in Krit1^{ECKO} compared to Krit1^{fl/fl} BMEC controls (S.E.M., N=3 or 4). FIG. 2c, Confocal microscopy of cerebellar cortex at P7 stained with anti-PECAM1 (green). FIG. 2d, Higher magnification images of boxed areas in ε stained for ZO1 (red), claudin5 (turquoise) and PECAM1 (green). Arrow indicates staining of tight junction proteins, ZO1 and claudin5 (N=3). FIG. 2e, Quantification of brain endothelial ZO1, claudin5 and VE-cadherin protein abundance in freshly isolated cerebellar microvasculature in Krit1^{ECKO} compared to Krit1^{fl/fl} littermate controls (S.E.M., N=3 or 4). FIG. 2f, Maximum intensity projection of whole-mount P7 retinal vasculature at the angiogenic growth front stained for ZO1 (red), claudin5 (turquoise) and an endothelial marker, Isolectin B4 (green). FIG. 2g, Higher magnifications images of boxed areas in f show staining for ZO1 (red), claudin5 (turquoise), and Isolectin B4 (green). FIG. 2h, Quantification of ZO1 and claudin5 protein expression in retinal vasculature at the angiogenic front in Krit1^{ECKO} compared to Krit1^{fl/fl} littermate controls (S.E.M., N=6 mice per group). Scale bars, 50 μm FIG. 2a, 100 μm FIG. 2c, 25 μm FIG. 2d, 25 μm FIG. 2f, 25 μm FIG. 2g. *P<0.05, **P<0.01.

FIGS. 3a-3h. Reconstitution of TSP1 prevents the tight junction loss that follows inactivation of Krit1. FIG. 3a, Five nM mouse recombinant TSP1 (mrTSP1) was added to cultured Krit1^{ECKO} BMEC 72 h after initial treatment with 4-hydroxy-tamoxifen. Note continuous ZO1 junctional staining in TSP1-treated Krit1^{ECKO} BMEC (Arrowheads), resembling the appearance of Krit1^{fl/fl} BMEC. In sharp contrast, ZO1 staining was reduced and discontinuous (arrows) in vehicle-treated Krit1^{ECKO}. FIG. 3b, Treatment with an anti-angiogenic domain (20 nM), 3-thrombospondin1 type 1 repeats (3TSR). The arrows in Krit1^{ECKO} BMEC indicate immunostaining for ZO1 that is punctate and reduced at the cell-cell contacts. Treatment with 3TSR prevented loss of ZO1 protein from tight junctions in Krit1^{ECKO} BMEC (arrowheads). FIG. 3c, Quantification of ZO1 protein expression in BMEC. Krit1^{ECKO} or control Krit1^{f4} BMEC treated with TSP1, 3TSR or vehicle as indicated (S.E.M., N=3). FIG. 3d, ZO1 protein levels as determined by Western blot analysis in Krit1^{ECKO} and Krit1^{fl/fl} BMEC in presence or absence of 20 nM 3TSR (S.E.M., N=3). FIG. 3e, Quantification of ZO1 protein expression in cerebellar tissue in Krit1^{ECKO} and control Krit1^{fl/fl} mice treated with 3TSR or vehicle (S.E.M., N=4 mice in each group). FIG. 3f, VEGFR2-Tyr¹¹⁷⁵ phosphorylation in primary BMEC Krit1^{ECKO} or control Krit1^{fl/fl} BMEC treated with 3TSR or vehicle. Nuclei were counterstained with DAPI (blue). FIG. 3g, Quantification of VEGFR2-Tyr¹¹⁷⁵ phosphorylation in BMEC is shown as integrated density in Krit1^{ECKO} and Krit1^{fl/fl} controls in presence or absence of 20 nM 3TSR (S.E.M., N>47 cells). FIG. 3h, Quantification of VEGFR2-Tyr¹¹⁷⁵ phosphorylation in cerebellar tissue in Krit1^{ECKO} and control Krit1^{fl/fl} treated with 3TSR or vehicle 30 min after VEGF treatment (75 μg/Kg), as assessed by Western Blot analysis (S.E.M., N=4 mice in each group). Scale bar, 50 μm in FIG. 3a and FIG. 3b. *P<0.05, **P<0.01, ***P<0.001 vs vehicle treated Krit1^{ECKO}, ##P<0.01, ###P<0.001 vs vehicle treated Krit1^{fl/fl}.

FIGS. 4a-4c. TSP1 limits CCM formation in Krit1^{ECKO} mice. FIG. 4a, Prominent lesions are present in the cerebellum of Krit1^{ECKO} mice whereas administration of 3TSR suppressed lesion formation. Increased CCM lesions were observed in Krit1^{ECKO}; Thbs1^{+/-} and Krit1^{ECKO}; Thbs1^{+/-} mice. FIG. 4b, Quantification of lesion volumes by microCT analysis from mice in experiment depicted in panel FIG. 4a. Control were either vehicle-treated mice or untreated mice which had similar lesion volumes. All groups were compared to control Krit1^{ECKO} mice (S.E.M., N>16 mice in each group, except Krit1^{ECKO}; Thbs1^{+/-} mice⁻ N=3). FIG. 4c, Survival of Krit1^{ECKO} and Krit1^{ECKO}; Thbs1^{+/-} mice. The numbers in parentheses indicate the number of mice in each group. Statistical significance was analyzed by log-rank test for comparing the survival rates. *P<0.05, *** P<0.001.

FIGS. 5a-5i. TSP1 replacement does not suppress the rise in KLF2 and KLF4 following loss of KRIT1. FIGS. 5a, 5b, Analysis of TSP1, and ZO1, KLF2 and KLF4, mRNA levels by RT-qPCR in freshly isolated microvasculature from mice at P5 and P7 as indicated. Krit1^{+/-} littermate controls, at each developmental stage, were used to calculate % increase or decrease in Krit1^{ECKO} mice using the formula: % increase=100*(X−F)/F and % decrease=100*ABS((F−X)/F) where X and F=mRNA abundance in F=Krit1^{fl/fl} or X=Krit1^{ECKO} BMEC (S.E.M., n=4 or 6). FIG. 5c, Representative confocal images of retinal vasculature stained for KLF4 (green), TSP1 (red), or with isolectin B4 (turquoise). TSP1 is decreased and KLF4 is increased at areas of condensed vasculature (N=5 or 6 mice in each group). FIGS. 5d, 5e, Analysis of levels of KLF2 and KLF4 mRNA by RT-qPCR from Krit1^{ECKO} BMEC (d) or cerebellar tissue from Krit1^{ECKO} mice(e) treated with 3TSR, TSP1, or Vehicle compared to Krit^{fl/fl} BMEC or Krit1^{fl/fl} controls. Data is expressed as % increase or decrease in Krit1^{ECKO} using the formula: % increase=100*(X−F)/F and % decrease=100*ABS((F−X)/F) where X and F=mRNA abundance in F=Krit1^{fl/fl} or X=Krit1^{ECKO} BMEC respectively (S.E.M., N=3 or 4 in each group). FIG. 5f, HUVECs were transduced with lentivirus encoding shKrit1, KLF2, or KLF4 and the increase in KLF2 or KLF4 mRNA relative to cells transduced with lentivirus encoding EGFP was measured by RT-qPCR. (S.E.M., N=4). FIG. 5g, HUVECs were transduced with lentivirus encoding ShKrit1, KLF2, or KLF4 as described in panel FIG. 5f and the decrease of TSP1 mRNA levels were measured relative to cells transduced with EGFP control lentivirus (S.E.M., N=4 or 5). FIG. 5h, Analysis of TSP1 protein levels in HUVECs transduced with lentivirus encoding KLF2 or KLF4 as assessed by Western Blot analysis, lentivirus encoding GFP was used as a controls (S.E.M., N=4). FIG. 5i, Loss of endothelial KRIT1 increases expression of KLF2 and KLF4 transcription factors contributing to CCM formation by downstream effects including suppressed TSP1 expression. 3TSR (TSP1 derivative) reduces CCM lesion formation by replacing functions of TSP1 such as blocking VEGF signaling. Loss of KRIT1 also leads to ROCK activation in a KLF2 dependent manner and blocking ROCK can also ameliorate CCMs. Thus, blockade of these and other downstream targets of KLF2 and KLF4 may offer a general strategy to reduce CCM formation in humans Scale bar is 25 m in FIG. 5c, *P<0.05, ** P<0.01.

FIGS. 6a-6e. Acute genetic inactivation of brain endothelial Krit1. FIG. 6a, Protocol for acute genetic inactivation of Krit1 in primary brain microvascular endothelial cells (BMEC) from Pdgfb-iCreERT2; Krit1^{fl/fl} (Krit1^{ECKO}) or control Krit1^{fl/fl} mice. FIG. 6b, Quality control of BMEC. RT-qPCR analysis of mRNA of EC-specific genes (Pecaml and VE-cadherin) and those expressed by potential contaminating cells (Cd45, Pdgfr, Gfap) (S.E.M., N=4). FIG. 6c, Confirmation of deletion: Krit1. Krit1^{fl/fl}; Pdgff-Cre-ER(T) (lane 1 and 2) or Krit1^{fl/fl} (Lane 3 and 4) BMEC were treated with 5 (Lane 1 and 3) or 0.5 (Lane 2 and 4) M 4-hydroxy-tamoxifen and analyzed by PCR using primers that selectively amplify the deleted allele (Krit1KO) or primers that amplify the floxed allele (Krit1Flox). Lane 5 is a water blank. FIG. 6d and FIG. 6e, Quantification of KRIT1 mRNA and protein levels using RT-qPCR FIG. 6d and a previously described³⁶ tandem ELISA FIG. 6e respectively (S.E.M., N=4). ***P<0.001.

FIGS. 7a-7d. RNA-seq analysis of BMEC transcriptome following acute genetic inactivation of Krit1. FIG. 7a, Distribution of raw counts are shown for Krit1^{ECKO} and Krit1^{fl/fl} BMEC FIG. 7b, Irreproducibility discovery rate (IDR) analysis. Genes, represented by dots between samples, are noted as reproducible (black) or irreproducible (red). Irreproducible genes were excluded from further analysis. FIG. 7c, List of the top 100 differentially expressed genes in BMEC from Krit1^{ECKO} compared to BMEC from Krit1^{fl/fl}. FIG. 7d, Validation of RNA-seq findings by RT-qPCR. Levels of 9 selected genes. Krit1^{fl/fl}, controls were normalized to one and results are expressed as relative mRNA levels in Krit1^{ECKO} BMEC. Actin-β was used as an internal standard (S.E.M., N=3).

FIGS. 8a-8c. Acute genetic inactivation of endothelial Krit1 in vivo. FIG. 8a, Protocol for genetic inactivation of Krit1 in vivo follow by sacrifice at P5 and P7-P10 in Krit1^{ECKO} or control Krit1^{fl/fl} mice. FIG. 8b, Quality control of freshly isolated brain microvasculature: RT-qPCR analysis of mRNA of EC-expressed gene (Pecaml) and those expressed by potentially contaminating cells (Cd45, Gfap, Pdgfr). Results are expressed as mRNA relative abundance. FIG. 8c, Hematoxylin and eosin staining of cerebellar sections from Krit1^{ECKO} and Krit1^{fl/fl} mice of regions imaged in FIGS. 1g, 1h. Scale bar is 200 μm.

FIGS. 9a-9c. Loss of KRIT1 decreased human endothelial TSP1. FIG. 9a, Immunofluorescent staining of TSP1 (red) and collagen IV (green) of human CCM and of lesion-free brain tissue. FIGS. 9b-9c, HUVECs were transduced with shKrit1 or shControl (shCtl) using lentivirus. FIG. 9b, KRIT1-depleted cells (˜55% reduction) leads to a ˜35% decrease in TSP1 mRNA levels as determined by RT-qPCR (S.E.M., N=4). FIG. 9c, KRIT1-depleted HUVEC expressed ˜50% as much TSP1 protein as control cells (S.E.M., N=3). Scale bars is 100 μm. **P<0.01, ***P<0.001.

FIG. 10. Acute loss of KRIT1 decreases tight junctions in BMEC. Representative confocal images of claudin5 (CLDN5) (turquoise) and VE-cadherin (green) staining in primary BMEC Krit1^{ECKO} or control Krit1^{fl/fl} BMEC. Note continuous VE-cadherin junctional staining is observed in Krit1^{ECKO} BMEC, in contrast claudin5 staining was reduced and discontinuous (Arrows), Nuclei were counterstained with DAPI (blue) (N=4). Scale bar is 100 μm.

FIGS. 11a-11d. Characterization of TSP1 and 3TSR proteins. FIG. 11a, Schematic of the experimental strategy wherein mouse recombinant TSP1 (mrTSP1) was added following genetic inactivation of Krit1 in cultured BMEC. FIG. 11b, The purity of the TSP1 was assessed by Ponceau staining and Western blotting. FIG. 11c, Schematic structure of TSP1 and the anti-angiogenic domain, 3-thrombospondin1 type 1 repeats (3TSR). FIG. 11d, The purity of the 3TSR was assessed by Ponceau staining and Western blotting.

FIGS. 12a-12g. TSP1 derivative, 3TSR, prevents CCMs and retinal vascular lesions in Krit1^{ECKO} mice. FIG. 12a, Experimental protocol: Vehicle or 3TSR (1.6 mg/Kg) were administered by retro-orbital plexus injection at P5 and P6 and brains and retinas were analyzed at P7. FIG. 12b, Prominent hemorrhagic lesions are present in the cerebellum of Krit1^{ECKO} mice whereas administration of 3TSR suppressed lesion formation. FIG. 12c, Hematoxylin and eosin staining of cerebellar sections from Krit1^{ECKO} mice after treatment with 3TSR or Vehicle (N=4). FIG. 12d, Representative image of whole-mount P7 retinal vasculature at the angiogenic growth front. The arrows in Krit1^{ECKO} whole-mount retina show decreased areas of condensed vascular plexus in Krit1^{ECKO} treated with 3TSR when compared with vehicle-treated Krit1^{ECKO} littermates (S.E.M., N=8 mice in each group). FIG. 12e, Quantification of lesion coverage in Krit1^{ECKO} mice treated with 3TSR or Vehicle (S.E.M., N=8 mice in each group). FIG. 12f, Administered 3TSR is present in CCM. 3TSR was injected retro-orbitally into a Krit1^{ECKO}; Thbs11-mouse and after 30 min, the mouse was sacrificed and its cerebellar cortex was stained for 3TSR (red, using anti-TSP1 antibodies) and an endothelial marker PECAM1 (green), DAPI staining (blue) was used to reveal nuclei. 3TSR is observed in CCM vascular lesions (arrowheads) whereas it is absent in nearby brain vasculature (arrows). FIG. 12g, Higher magnification images of boxed areas in FIG. 12f. Scale bars, 1 mm FIG. 12b, 100 μm c, 200 μm FIG. 12d, 100 μm e, 50 μm FIG. 12f. *** P<0.001 vs vehicle treated Krit1^{ECKO} mice.

FIGS. 13a-13d. 3TSR prevents VEGFR2-Tyr¹¹⁷⁵ phosphorylation in human endothelial cells. FIGS. 13a-13b HUVECs were transduced with shKrit1 or shControl (shCtl, control cells) using lentivirus. Cell monolayers were pretreated for 5 h with 20 nM 3TSR in low-serum medium before adding 50 ng/ml VEGF for 10 min. pVEGFR2-Tyr¹¹⁷⁵ protein levels were determined by Western Blot FIG. 13a and quantification of pVEGFR2-Tyr¹¹⁷⁵ protein expression in HUVEC ShKRIT1 or ShControl treated with 3TSR or vehicle. VEGFR2 and Actin were used as loading controls (S.E.M., N=4). FIG. 13c, 2 μM SU5416 (VEGFR2 inhibitor) was added to cultured Krit1^{ECKO} BMEC 72 h after initial treatment with 4-hydroxy-tamoxifen. Note continuous ZO1 junctional staining in SU5416-treated Krit1^{ECKO} BMEC (Arrowheads), resembling the appearance of Krit1^{fl/fl} BMEC. In sharp contrast, ZO1 staining was reduced and discontinuous (arrows) in vehicle-treated Krit1. Scale bars is 50 m c. *P<0.05, vs vehicle treated Krit1^{ECKO}. One-tailed unpaired Student's t-test.

FIGS. 14a-14d. 3TSR do not activate TGFb signaling in Krit1^{ECKO}. FIGS. 14a-14b, Analysis of levels of TGFb target genes, Crebbp, Mcp1, Pail mRNA by RT-qPCR from BMEC Krit1^{ECKO} treated with 3TSR, TSP1, or Vehicle FIG. 14a and from cerebellar tissue from Krit1^{ECKO} mice after treatment with 3TSR or Vehicle FIG. 14b. Results are expressed as mRNA relative levels to control Krit1/fl vehicle treated. FIG. 14c, pSMAD3 and SMAD3 protein levels as determined by Western blot analysis in Krit1^{ECKO} and Krit1^{fl/fl} BMEC in presence or absence of 20 nM 3TSR.

### DETAILED DESCRIPTION OF THE INVENTION

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Pharmaceutically active: The term “pharmaceutically active” as used herein refers to the beneficial biological activity of a substance on living matter and, in particular, on cells and tissues of the human body. A “pharmaceutically active agent” or “drug” is a substance that is pharmaceutically active and a “pharmaceutically active ingredient” is the pharmaceutically active substance in a drug. As used herein, pharmaceutically active agents include synthetic or naturally occurring small molecule drugs and more complex biological molecules.

Pharmaceutically acceptable: The term “pharmaceutically acceptable” as used herein means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

Pharmaceutically acceptable salt: The term “pharmaceutically acceptable salt” as used herein refers to acid addition salts or base addition salts of compounds, such as a thrombospondin 1 protein agent, in the present disclosure. A pharmaceutically acceptable salt is any salt which retains the activity of the parent compound and does not impart any deleterious or undesirable effect on a subject to whom it is administered and in the context in which it is administered. Pharmaceutically acceptable salts may be derived from amino acids including, but not limited to, cysteine. Methods for producing compounds as salts are known to those of skill in the art (see, for example, Stahl et al., Handbook of Pharmaceutical Salts: Properties, Selection, and Use, Wiley-VCH; Verlag Helvetica Chimica Acta, Ziurich, 2002; Berge et al., J Pharm. Sci. 66: 1, 1977). In some embodiments, a “pharmaceutically acceptable salt” is intended to mean a salt of a free acid or base of a compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

Pharmaceutically acceptable carrier: The terms “pharmaceutically acceptable carrier” as used herein refers to an excipient, diluent, preservative, solubilizer, emulsifier, adjuvant, and/or vehicle with which a compound, such as a thrombospondin 1 protein agent, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

As used herein, “preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition.

As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition. More specific embodiments are included in the sections below. In some embodiments, the term “therapeutically effective amount” or “effective amount” refers to an amount of a therapeutic agent that when administered alone or in combination with an additional therapeutic agent to a cell, tissue, or subject is effective to prevent or ameliorate the disease or condition such as an infection or the progression of the disease or condition. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

As used herein, the term “combination” refers to either a fixed combination in one dosage unit form, or a kit of parts for the combined administration where a compound and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein, a subject in need refers to an animal, a non-human mammal or a human. As used herein, “animals” include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate, including a monkey and a chimpanzee.

Other objects, advantages and features of the present invention will become apparent from the following specification taken in conjunction with the accompanying drawings.

This disclosure generally provides pharmaceutical compositions, and methods of use thereof, for treating cerebral cavernous malformations and symptoms associated therewith in a subject. The pharmaceutical compositions generally include a thrombospondin 1 protein agent and the methods of treatment generally include administering a thrombospondin 1 protein agent to a subject in need thereof. The present pharmaceutical compositions and methods can be used to treat any suitable subject in need of treatment. In some embodiments, the subject is a mammal. In some embodiments, the mammal is a human.

In some embodiments, the thrombospondin 1 protein agent is a thrombospondin 1 protein, a functional fragment of thrombospondin 1 protein, a thrombospondin 1 protein isomer, a functional fragment of a thrombospondin 1 protein isomer, a homolog of thrombospondin 1 protein, a functional fragment of a homolog of thrombospondin 1 protein, a peptidomimetic of thrombospondin 1 protein or a functional fragment thereof, a small molecule mimic of thrombospondin 1 protein or a functional fragment thereof, or a combination thereof. The thrombospondin 1 protein agent can also be a pharmaceutically acceptable salt of the foregoing. Other thrombospondin 1 protein agents suitable for use with the present disclosure will be readily appreciated by those of ordinary skill in the art.

As used herein, the term “thrombospondin 1 protein agent” refers to and includes any isolated or purified native or recombinant thrombospondin 1 protein, homolog, isomer, peptidomimetic, functional fragment or motif, and/or mutant thereof. The term “thrombospondin 1 protein agent” also refers to any small molecule mimic of thrombospondin 1 protein or a functional fragment thereof. Examples include thrombospondin 1, 3TSR, and ABT-510. In some embodiments, the functional fragments and homologs of thrombospondin 1 protein exhibit at least 20%, 30%, 40%, 50%, 60%, 70%, 80%, 90%, or 95% of the therapeutic effect in treating cerebral cavernous malformations of thrombospondin 1 protein itself. In some embodiments, the thrombospondin 1 protein agent comprises an amino acid sequence for a human native or recombinant thrombospondin 1 protein. In some embodiments, the thrombospondin 1 protein agent shares the primary amino acid structure of any known thrombospondin 1 protein or isoform with at least 60% homology, preferably 75% homology, more preferably 85%, 90%, 95%, 96%, 97%, 98%, 99% or more homology.

In some embodiments, the thrombospondin 1 protein agent comprises a biologically active portion of the thrombospondin 1 protein. As used herein, a “biologically active portion” of a protein includes a functional fragment of the protein comprising amino acid sequences sufficiently homologous to, or derived from, the amino acid sequence of the protein, which includes fewer amino acids than the full length protein, and exhibits at least one activity of the full-length protein, i.e., an ability to treat CM in a subject. Typically a biologically active portion comprises a functional domain or motif with at least one activity of the protein. A biologically active portion of a protein can be a polypeptide which is, for example, 10, 25, 50, 100, 200, or more amino acids in length. In one embodiment, a biologically active portion of the thrombospondin 1 protein can be used as a therapeutic agent alone or in combination with other therapeutic agents for treating cerebral cavernous malformations.

In some embodiments, a therapeutically effective amount of a thrombospondin 1 protein agent is administered to a subject in need thereof in a therapeutically effective dosing regimen. A therapeutically effective amount of a particular thrombospondin 1 protein agent, and its therapeutically effective dosing regimen, will be appreciated by those of ordinary skill in the art.

In some embodiments, the methods for treating cerebral cavernous malformations comprise orally administering to a subject in need thereof a pharmaceutical composition including a thrombospondin 1 protein agent.

In some embodiments, the methods for treating cerebral cavernous malformations include administering a pharmaceutical composition including a thrombospondin 1 protein agent on a monthly, weekly, or daily administration regimen. In some embodiments, the methods for treating cerebral cavernous malformations include administering a dose of a pharmaceutical composition including a thrombospondin 1 protein agent one or more times a day. In some embodiments, the methods for treating cerebral cavernous malformations include administering a dose of a pharmaceutical composition including a thrombospondin 1 protein agent one or more times a week. In some embodiments, the methods for treating cerebral cavernous malformations include administering a dose of a pharmaceutical composition including a thrombospondin 1 protein agent one or more times a month.

In some embodiments, the methods for treating cerebral cavernous malformations include screening a subject, identifying the subject as having or being at risk for the development or progression of cerebral cavernous malformations, and then administering a therapeutically effective amount of a thrombospondin 1 protein agent in a therapeutically effective dosing regimen. In some embodiments, the subject is screened for mutations in one or more of the following genes: KRIT1, CCM2, PCDC10, which are risk factors for and/or indicative of CCM disease. If the subject has one or more mutations in the genes KRIT1, CCM2, PCDC10, the subject can be administered a therapeutically effective amount of a thrombospondin 1 protein agent in a therapeutically effective dosing regimen.

In some embodiments, the methods for treating cerebral cavernous malformations include administering to a subject in need thereof a therapeutically effective amount of a pharmaceutical composition comprising a thrombospondin 1 protein agent and a therapeutically effective amount of a pharmaceutical composition comprising a Rho Kinase inhibitor. In some embodiments, the thrombospondin 1 protein agent and the Rho Kinase inhibitor are coadministered. In some embodiments, the methods for treating cerebral cavernous malformations include administering to a subject in need thereof a therapeutically effective amount of a pharmaceutical composition comprising both a thrombospondin 1 protein agent and a Rho Kinase inhibitor.

In some embodiments, administering a therapeutically effective amount of a thrombospondin 1 protein agent in a therapeutically effective dosing regimen to a subject in need thereof reduces the subject's risk of developing vascular lesions, prevents the subject from developing vascular lesions, reverts the subject's vascular lesions to a non-diseased state, or a combination thereof.

In some embodiments, the pharmaceutical compositions comprising a thrombospondin 1 protein agent, alone or in combination with other active ingredient(s), described herein may further comprise one or more pharmaceutically-acceptable excipients. A pharmaceutically-acceptable excipient is a substance that is non-toxic and otherwise biologically suitable for administration to a subject. Such excipients facilitate administration of a thrombospondin 1 protein agent, alone or in combination with other active ingredient(s), described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the various embodiments are sterile compositions. Pharmaceutical compositions may be prepared using compounding techniques known or that become available to those skilled in the art.

Sterile compositions are within the present disclosure, including compositions that are in accord with national and local regulations governing such compositions.

In some embodiments, the pharmaceutical compositions and thrombospondin 1 protein agent, alone or in combination with other active ingredient(s), described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. A thrombospondin 1 protein agent, alone or in combination with other active ingredient(s), described herein, and preferably in the form of a pharmaceutical composition, may be administered by a suitable route of delivery, such as oral, parenteral, rectal, nasal, topical, or ocular routes, or by inhalation. In some embodiments, the compositions are formulated for parenteral, intravenous or oral administration.

For oral administration, a thrombospondin 1 protein agent, alone or in combination with another active ingredient, may be provided in a solid form, such as a tablet or capsule, or as a solution, emulsion, or suspension. To prepare the oral compositions, a thrombospondin 1 protein agent, alone or in combination with other active ingredient(s), may be formulated to yield a dosage of, e.g., from about 0.01 to about 50 mg/kg daily, or from about 0.05 to about 20 mg/kg daily, or from about 0.1 to about 10 mg/kg daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compositions may be formulated for rectal administration as a suppository. For parenteral use, including intravenous, intramuscular, intraperitoneal, intranasal, or subcutaneous routes, a thrombospondin 1 protein agent, alone or in combination with other active ingredient(s), may be provided in sterile aqueous solutions or suspensions, buffered to an appropriate pH and isotonicity or in parenterally acceptable oil. Suitable aqueous vehicles can include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, a thrombospondin 1 protein agent, alone or in combination with other active ingredient(s), may be administered using, for example, a spray formulation also containing a suitable carrier.

For topical applications, a thrombospondin 1 protein agent, alone or in combination with other active ingredient(s), are preferably formulated as creams or ointments or a similar vehicle suitable for topical administration. For topical administration, a thrombospondin 1 protein agent, alone or in combination with other active ingredient(s), may be mixed with a pharmaceutical carrier at a concentration of about 0.1% to about 10% of drug to vehicle. Another mode of administering a thrombospondin 1 protein agent, alone or in combination with other active ingredient(s), may utilize a patch formulation to effect transdermal delivery.

In certain embodiments, the present disclosure provides pharmaceutical compositions comprising a thrombospondin 1 protein agent, alone or in combination with other active ingredient(s), and methylcellulose. In certain embodiments, methylcellulose is in a suspension of about 0.1, 0.2, 0.3, 0.4, or 0.5 to about 1%. In certain embodiments, methylcellulose is in a suspension of about 0.1 to about 0.5, 0.6, 0.7, 0.8, 0.9, or 1%. In certain embodiments, methylcellulose is in a suspension of about 0.1 to about 1%. In certain embodiments, methylcellulose is in a suspension of about 0.1, 0.2, 0.3, 0.4, 0.5, 0.6, 0.7, 0.8, 0.8, or 1%. In certain embodiments, methylcellulose is in a suspension of about 0.5%.

One of ordinary skill in the art may modify the formulations within the teachings of the specification to provide numerous formulations for a particular route of administration. In particular, a thrombospondin 1 protein agent, alone or in combination with other active ingredient(s), may be modified to render them more soluble in water or other vehicle. It is also well within the ordinary skill of the art to modify the route of administration and dosage regimen of a particular thrombospondin 1 protein agent, alone or in combination with other active ingredient(s), in order to manage the pharmacokinetics of the present compounds for maximum beneficial effect in a patient.

It should be understood that the foregoing relates to embodiments of the present disclosure and that numerous changes may be made therein without departing from the scope of the disclosure.

### EXAMPLES

In this Example, genome-wide transcriptome analysis of BMEC following acute Krit1 inactivation was performed, and a signature of mRNA changes primarily affecting genes involved in cardiovascular development is reported. A striking finding was the dramatic suppression of TSP1, a potent endogenous angiogenesis inhibitor that was ascribed to KLF2 and KLF4-mediated repression of TSP1. Reduced TSP1 expression contributes to the pathogenesis of CCMs because loss of one or two copies of the gene encoding TSP1 (Thbs1) exacerbated CCM formation. Replenishing TSP1 in vitro with either full length TSP1 or 3TSR, an anti-angiogenic TSP1 fragment, prevented the disruption of BMEC tight junctions, an early phenotypic result of Krit1 inactivation. Administration of 3TSR prevented disruption of intercellular junctions due in part to the capacity of TSP1 or 3TSR to inhibit VEGF signaling. Administration of 3TSR inhibited formation of CCMs in Krit1^{ECKO} mice. Thus, loss of expression of TSP1, a key angiogenic checkpoint, plays an important role in pathogenesis of CCM and repurposing 3TSR or other relatively non-toxic TSP1 fragments or mimetics, provides a new approach to inhibit CCM development.

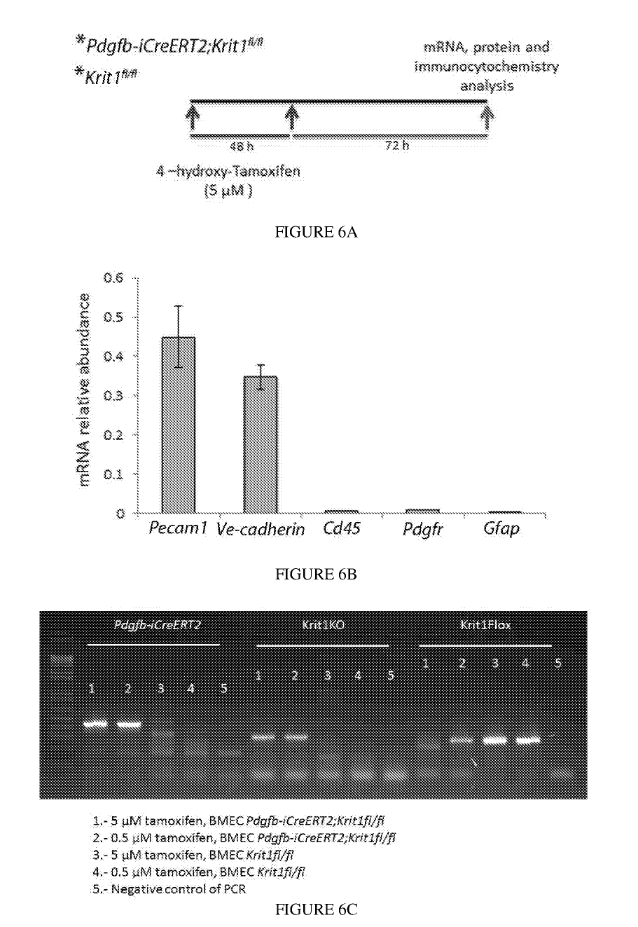

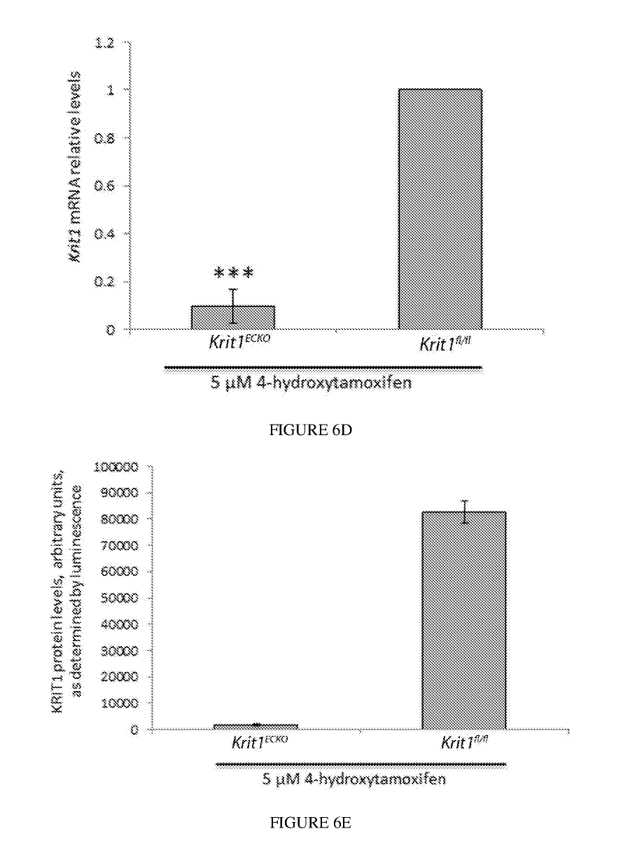

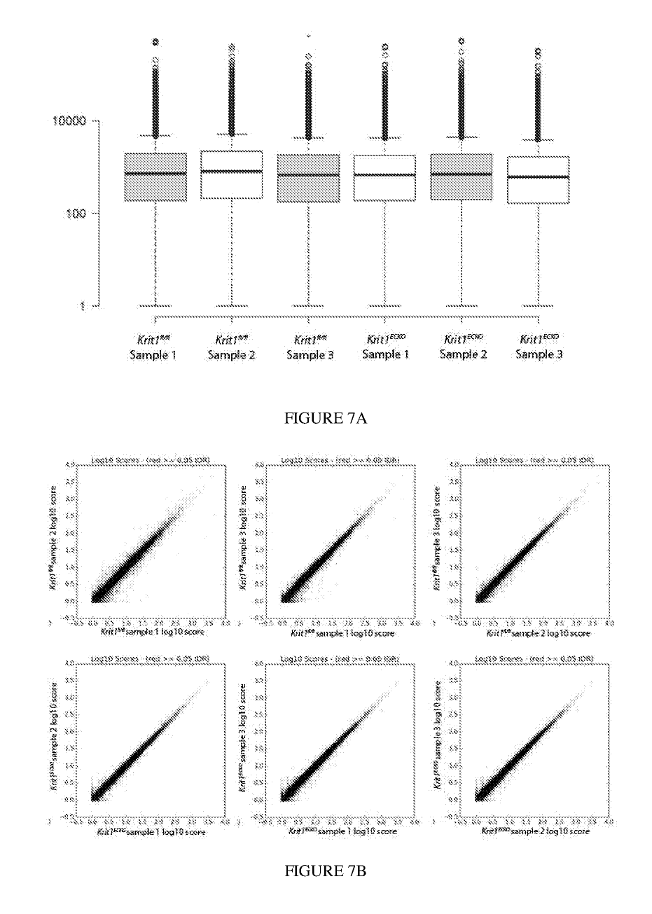

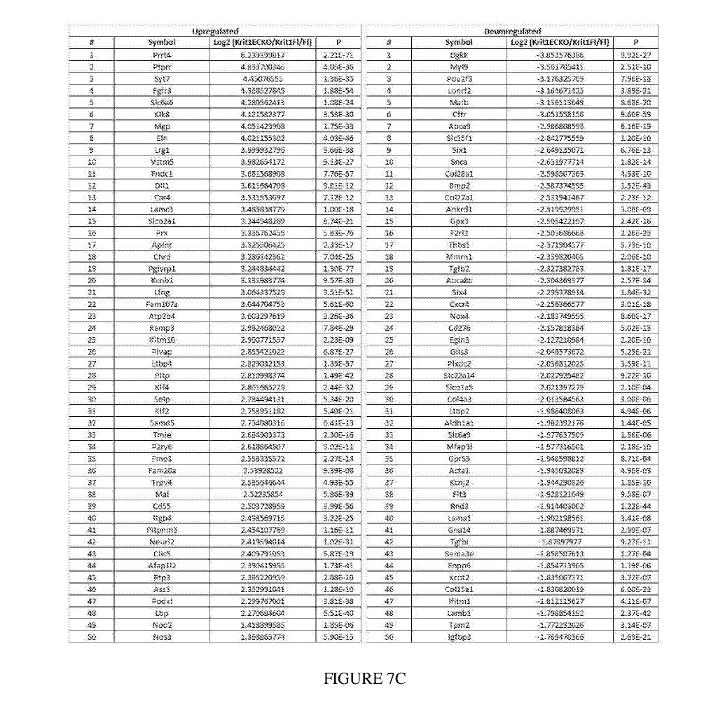

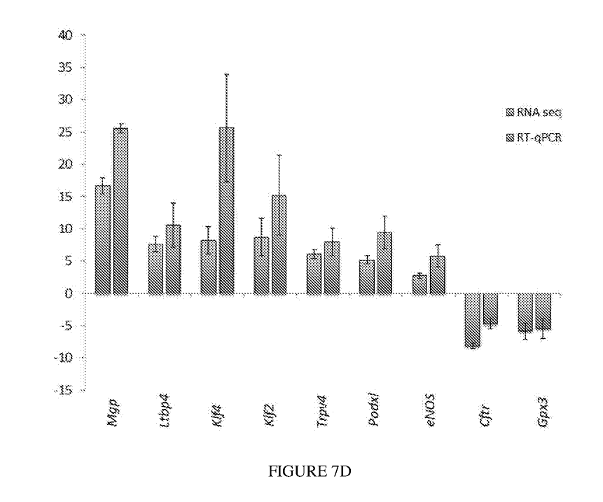

Genetic inactivation of Krit1 inhibits expression of thrombospondin1. To elucidate the pathogenesis of cerebral cavernous malformations (CCM) genome-wide RNA sequencing (RNA-seq) was used to characterize the transcriptome of primary brain microvascular endothelial cells (BMEC) following acute genetic inactivation of Krit1 (FIGS. 6a-6e). BMEC were isolated from mice bearing floxed alleles of Krit1 (Krit1^{fl/fl}) and an endothelial specific tamoxifen-regulated Cre recombinase (Pdgfb-iCreERT2)¹⁸. Treatment of Krit1^{f/f}-Pdgfb-iCreERT2 BMEC with 5 μM-hydroxy-tamoxifen deleted Krit1 (Krit1^{ECKO}) reduced KRIT1 mRNA and protein by >90% within 5 days compared to hydroxy-tamoxifen-treated Krit1^{fl/fl} littermates (FIGS. 6a-6e). Deep sequencing of cDNA from Krit1^{ECKO} and Krit1^{fl/fl} BMEC (FIGS. 7a-7d) revealed that acute loss of brain endothelial KRIT1 caused a dramatic change in gene expression in BMEC (FIGS. 7a-7d). This research identified 334 genes differentially expressed between the Krit1^{ECKO} and control Krit1^{fl/fl} (Corrected P<0.05, 2.5-fold change).

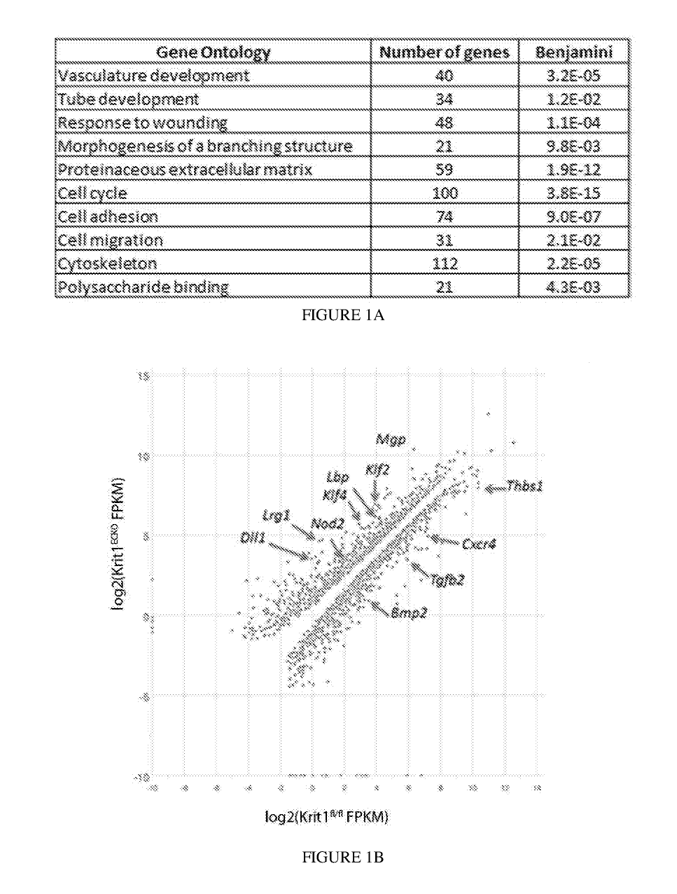

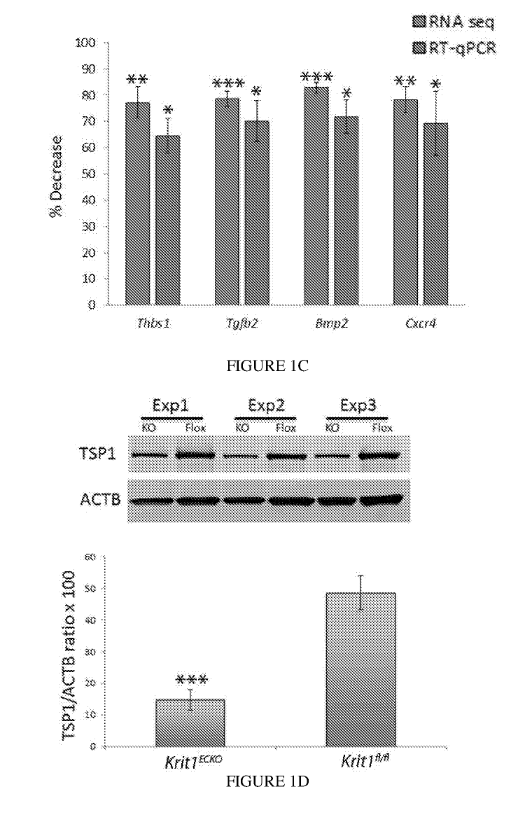

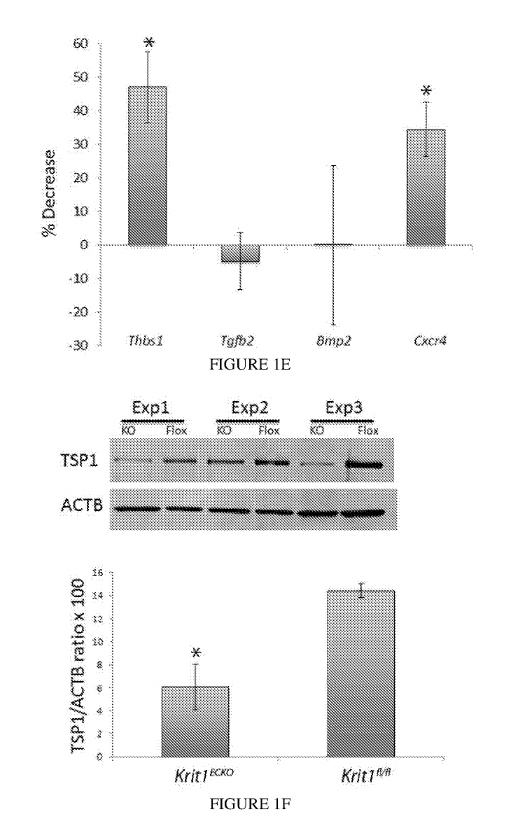

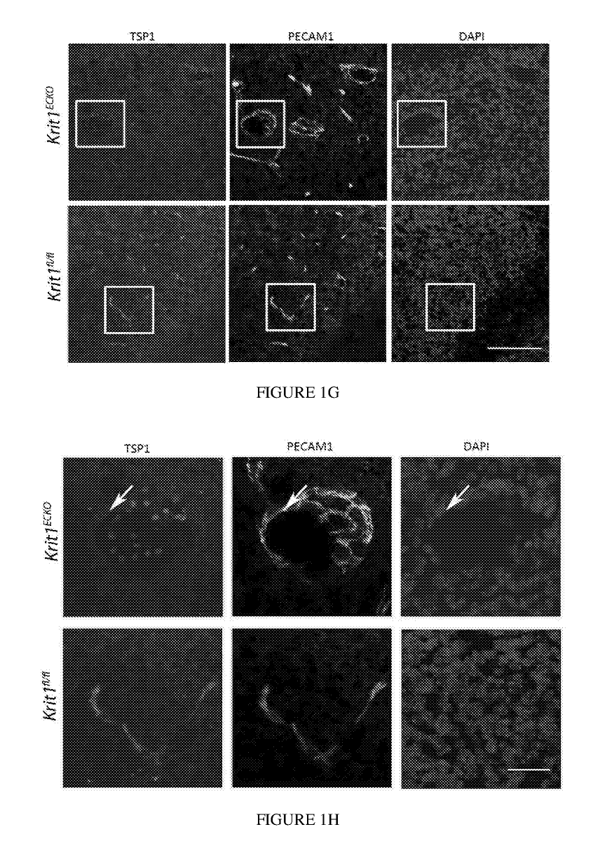

Gene Ontology analysis of the differentially-expressed genes indicated significant enrichment for terms related to cardiovascular development (P<3.2×10⁻⁵, FIG. 1a). Among genes known to be important in cardiovascular development were upregulation of Klf2 and Klf4, two transcription factors recently implicated in CCM pathogenesis^{7,8,11,12} (FIG. 1b, FIGS. 7a-7d). Among the most dramatic changes were a ˜75% decrease in expression of genes encoding secreted proteins or receptors that regulate angiogenesis, including Thbs1 (TSP1 is its protein product), Cxcr4, Bmp2, and Tgfb2 (FIG. 1b, 1c, FIGS. 7a-7d). The changes in TSP1 mRNA levels were associated with reduced TSP1 protein expression (˜3 fold decrease) in Krit1^{ECKO} BMEC (FIG. 1d).

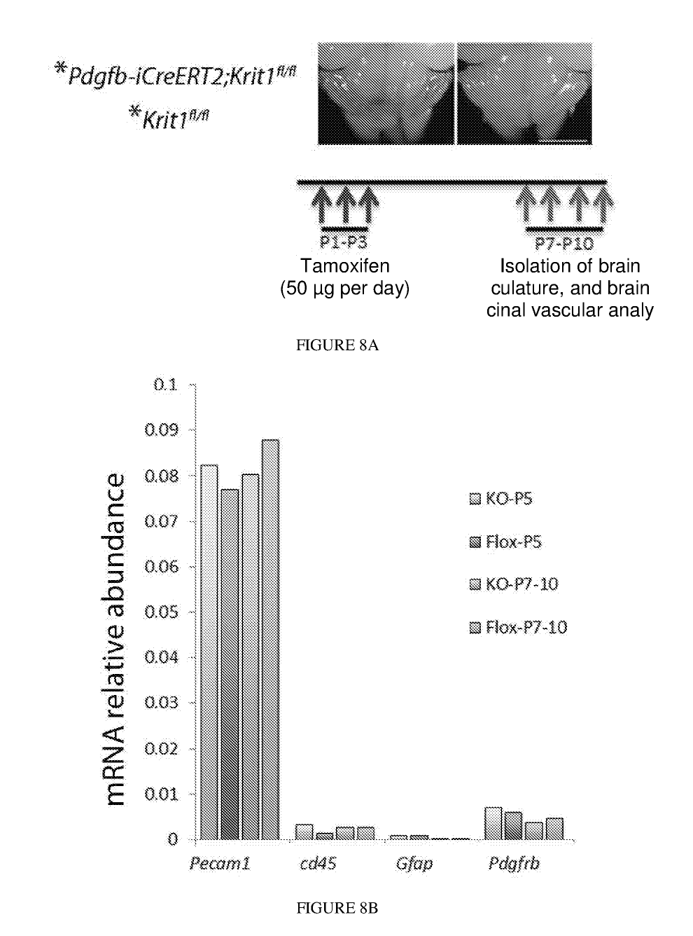

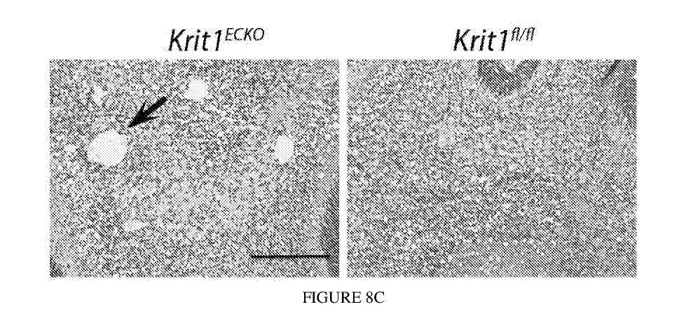

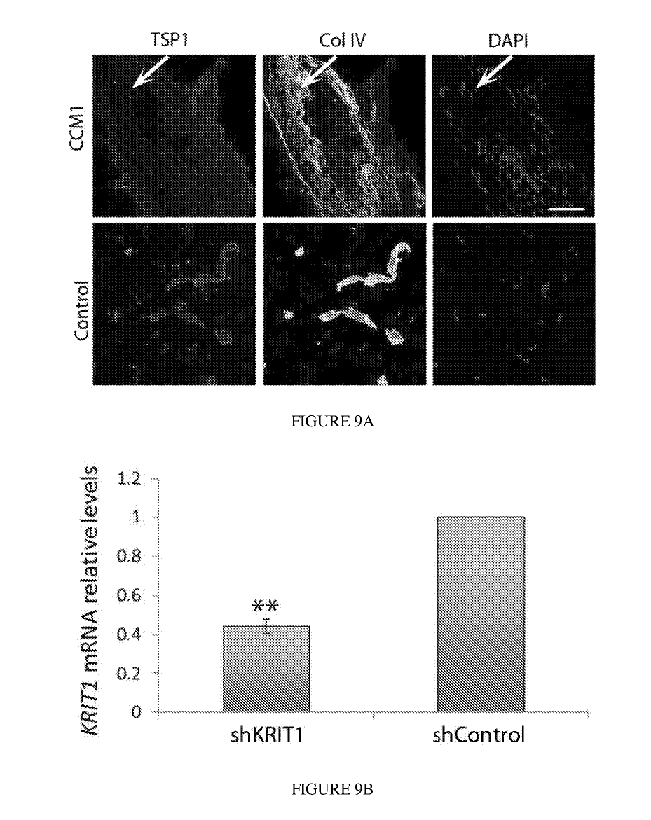

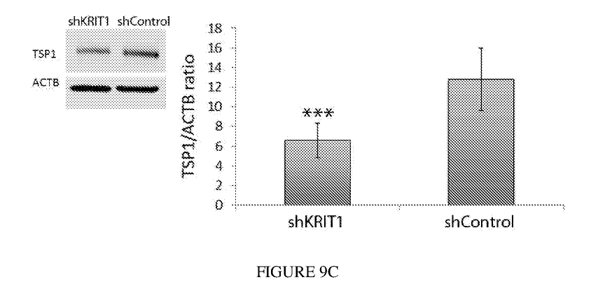

To confirm these reductions in potential extracellular regulators of angiogenesis in vivo, This research isolated brain microvasculature from tamoxifen-treated neonatal Krit1^{fl/fl}-Pdgfb-iCreERT2 (Krit1^{ECKO}) or Krit1^{fl/fl} mice (FIGS. 8a-8c) and quantified mRNAs with real time qPCR. Consistent with results observed in vitro, TSP1 and CXCR4 mRNA levels in freshly isolated brain microvasculature were reduced in P7 Krit1^{ECKO} mice (FIG. 1e). In sharp contrast, no significant changes were observed in BMP2 or TGFβ2 mRNA abundance (FIG. 1e). This research focused on TSP1 because reduced TSP1 mRNA expression was confirmed in vivo, TSP1 is an endogenous anti-angiogenic protein⁶, and TSP1 fragments and analogues have been developed as potential cancer therapeutic agents^{19,20}. This research confirmed that the reduction in mRNA was reflected in reduced TSP1 protein abundance in freshly-isolated brain microvasculature following genetic inactivation of endothelial Krit1 (FIG. 1f). Furthermore, there was a dramatic reduction in in situ TSP1 protein staining in CD31-positive endothelial cells in lesions of Krit1^{ECKO} mice in comparison to Krit1^{fl/fl} littermates (FIG. 1g, 1h). Loss of TSP1 expression also occurs during the pathogenesis of human CCM because silencing KRIT1 in human endothelial cells led to reduced TSP1 protein and mRNA expression (FIGS. 9a-9c). In addition, there was a dramatic decrease in endothelial TSP1 staining in human CCM lesions in comparison to a lesion-free brain tissue (FIGS. 9a-9c). Thus, inactivation of Krit1 in BMEC leads to early reductions in TSP1 mRNA and protein expression in vitro and in vivo, suggesting a role for the reduction of this secreted anti-angiogenic protein in the pathogenesis of CCM.

Altered Tight Junctions are an Early Event that Follows Loss of KRIT1.

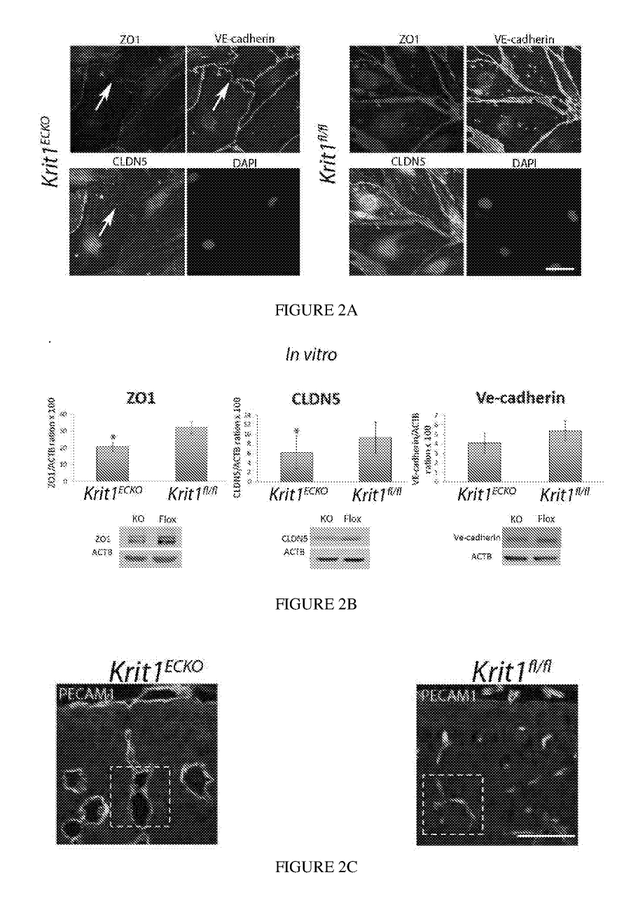

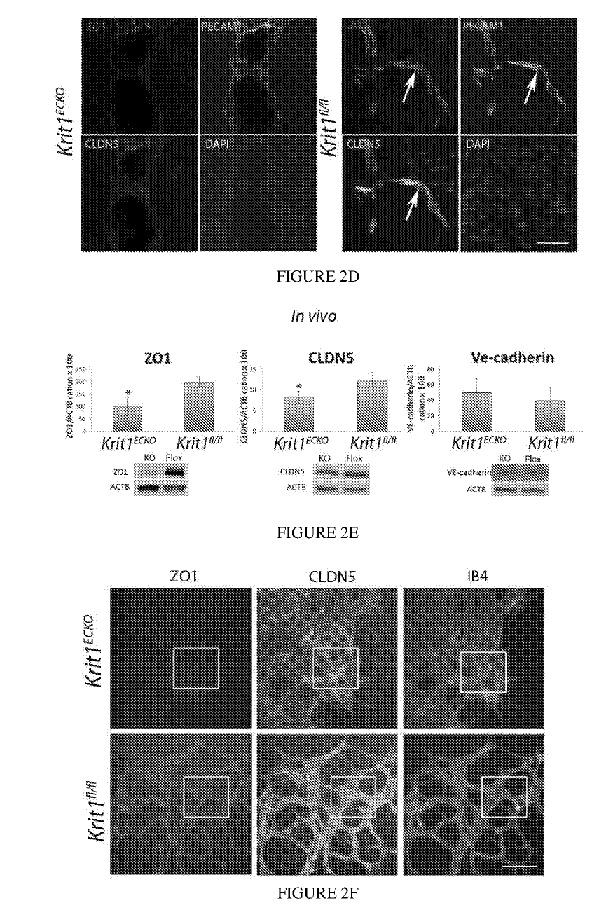

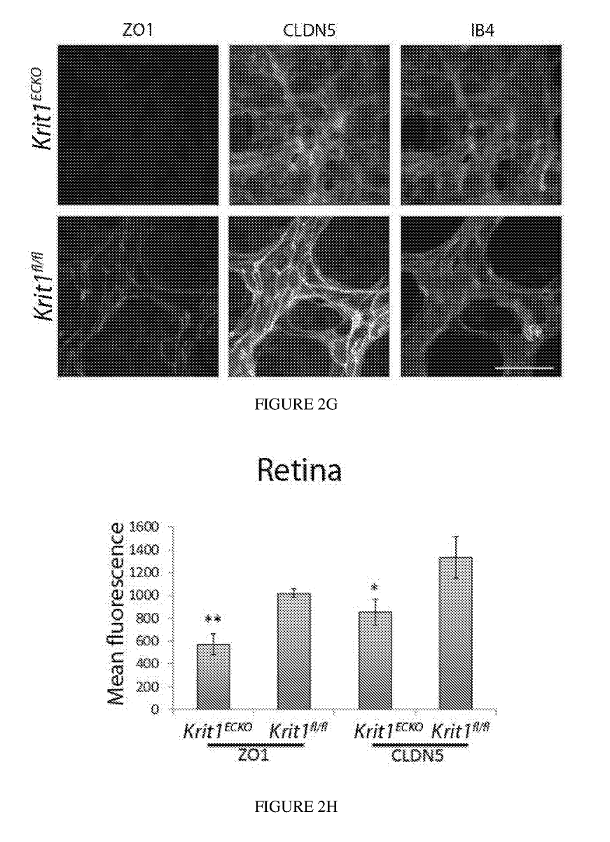

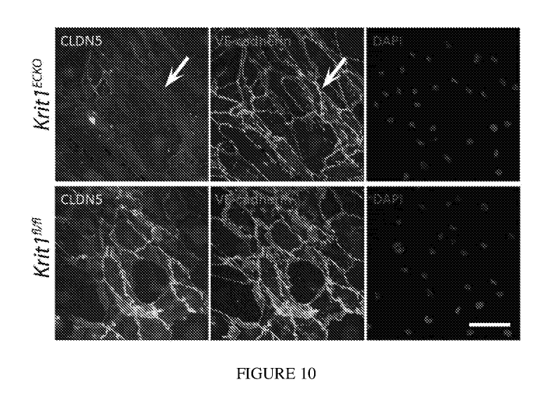

Because TSP1 is a large (˜450 kDa) glycoprotein, this research sought an in vitro intermediate phenotype to assess the effect of TSP1 reconstitution. Disruption of cell-cell junctions and increased vascular permeability are prominent features of CCM in humans^{5,21} and silencing of KRIT1 leads to disruption of intercellular junctions in human umbilical vein EC (HUVEC)²². This research examined the time course of altered cell-cell junctions in cultured BMEC following acute genetic inactivation of Krit1. There was striking loss of tight junction proteins ZO-1 and claudin-5 from BMEC junctions within 5 days of 4-hydroxy-tamoxifen treatment, an early time after the concentration of KRIT1 protein and mRNA were decreased (FIG. 2a and FIG. 10). Furthermore, immunoblotting revealed a 40-50% reduction in ZO-1 and claudin-5 protein abundance, following inactivation of Krit1 in BMEC (FIG. 2b). In contrast, at this early time point, the small decreases in VE-cadherin protein abundance and distribution were not statistically significant (FIGS. 2a, 2b). To assess whether similar changes were observed at early times in lesion evolution in vivo, this research examined the endothelial distribution of these junctional proteins in the brains of P7 Krit1^{ECKO} mice. ZO-1 and claudin-5 staining were markedly reduced in the dilated early CCM in these mice, whereas both tight junction proteins were abundant in normal vessels from Krit1^{ECKO} and Krit^{fl/fl} littermates (FIGS. 2c, 2d). Furthermore, there were reduced levels of ZO-1 and claudin-5 in microvasculature isolated from the cerebellum of P7-10 Krit1^{ECKO} mice as assessed by immunoblotting (FIG. 2e). This research did not observe significant changes in VE-cadherin protein levels in early lesions in Krit1^{ECKO} (P7-P10) mice (FIG. 2e). Similar changes were seen in the retinas of Krit1^{ECKO} mice (FIGS. 2f, 2g), a tissue that enables precise temporal and spatial assessment of vascular development²³. Collectively, these data show that changes in tight junctions, which are features of CCM, are early abnormalities that follow loss of KRIT1 in EC. Such changes could therefore represent an intermediate phenotype to assess potential interventions in CCM pathogenesis.

Reconstitution of TSP1 Prevents the Loss of Tight Junctions that Follows Inactivation of Krit1.

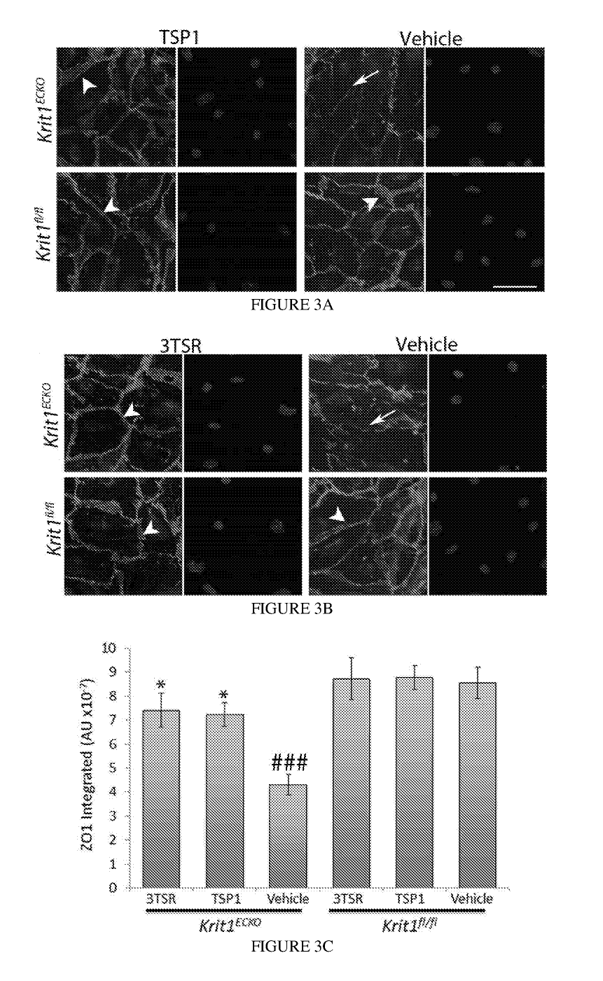

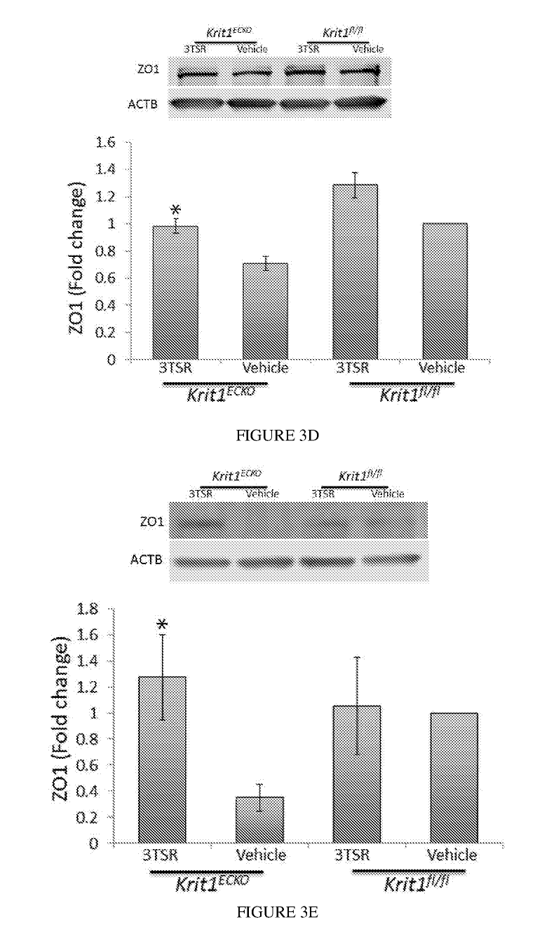

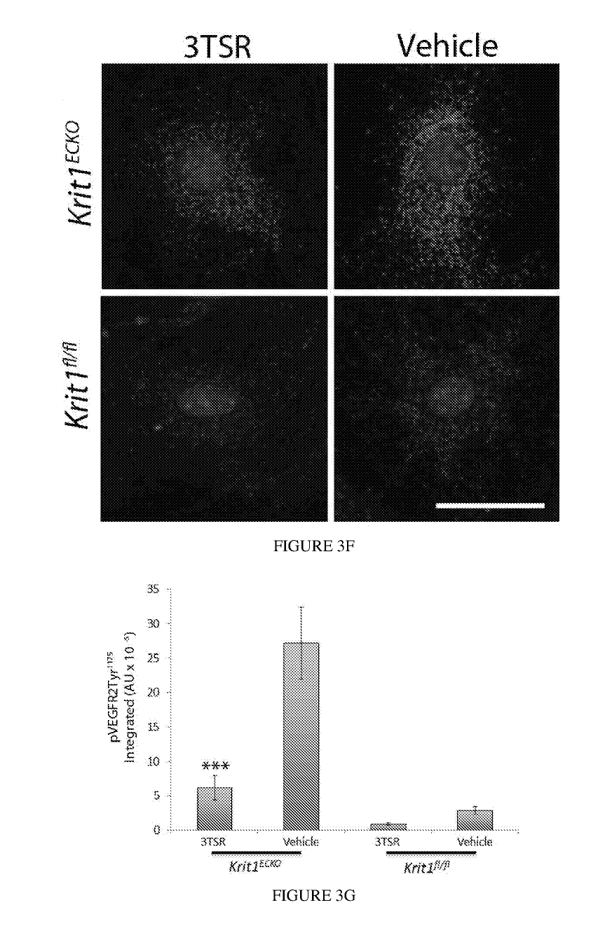

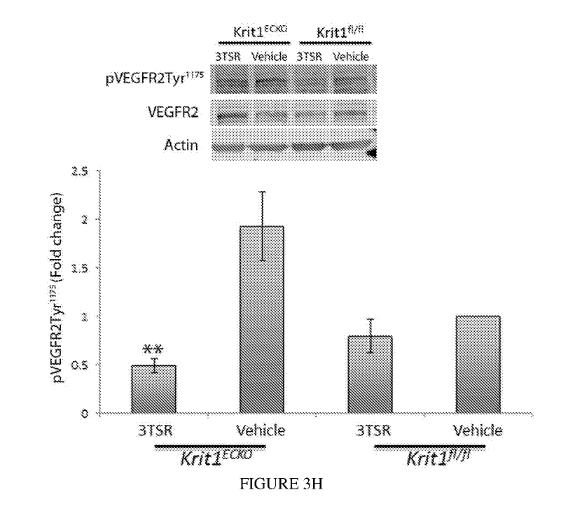

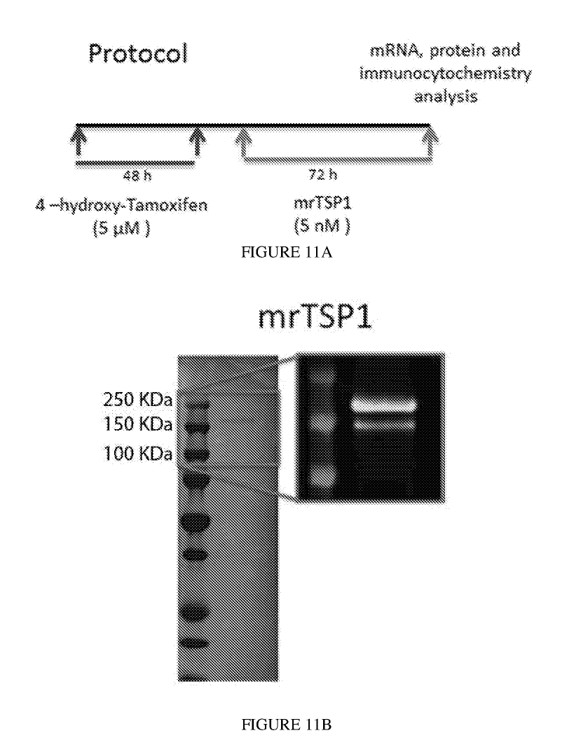

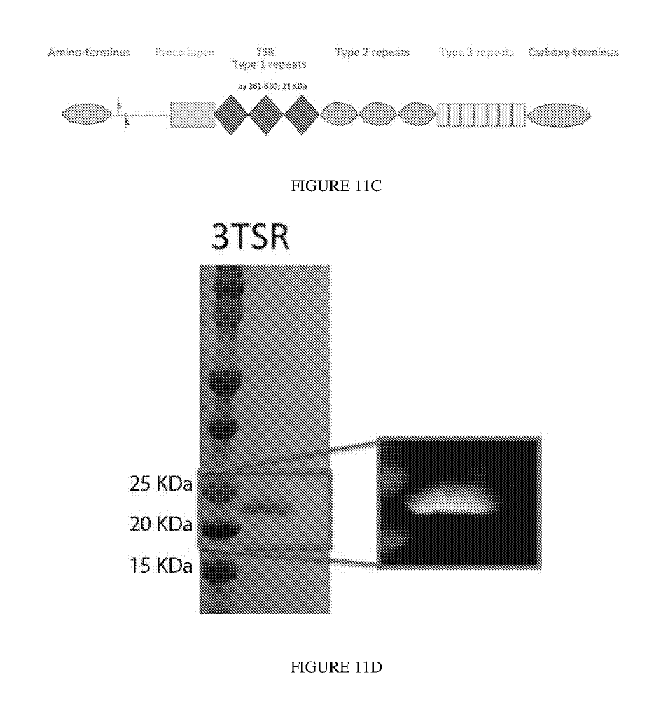

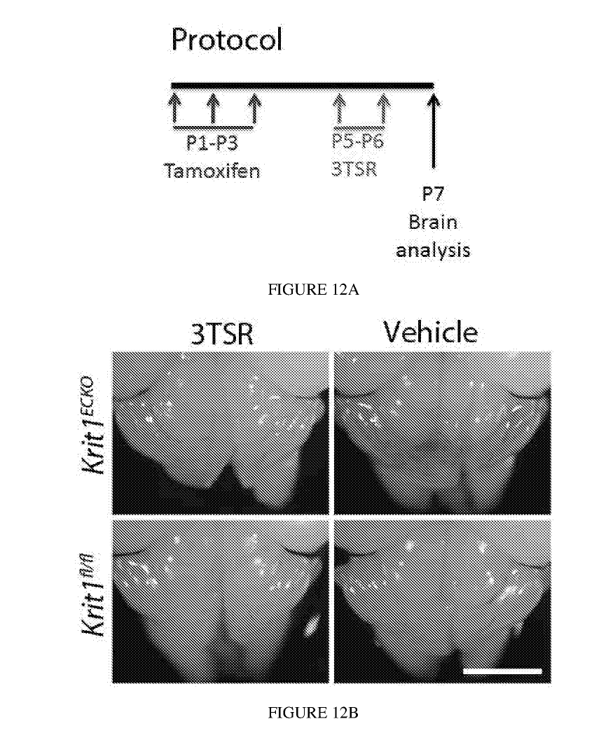

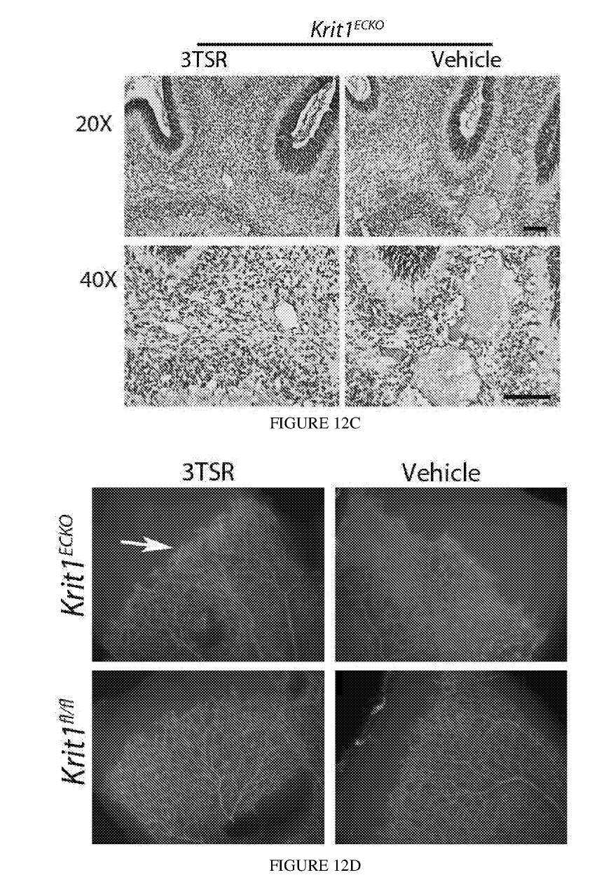

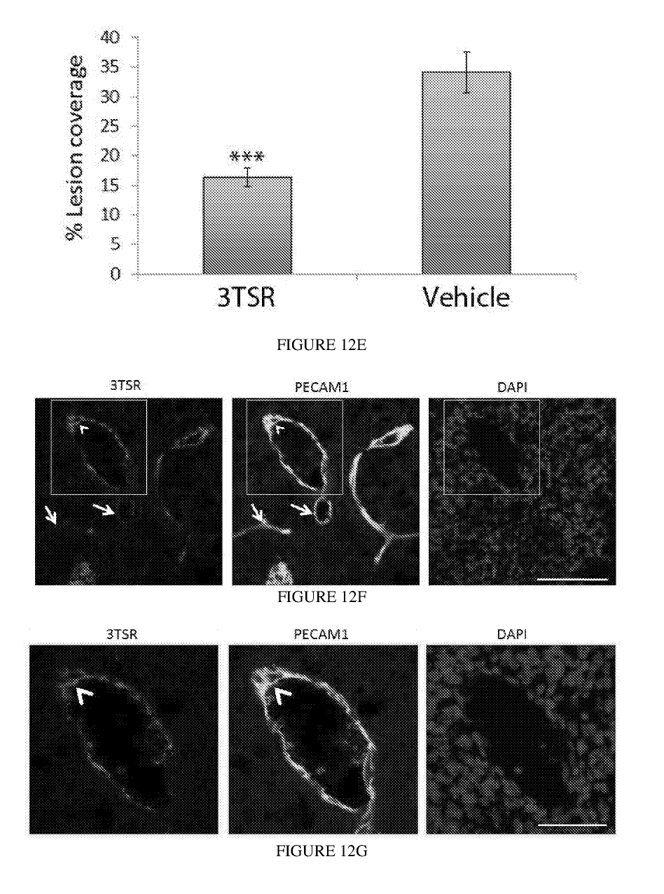

Having shown that altered tight junctions are an early result of loss of KRIT1 both in vitro and in vivo, this research assessed the effect of the addition of exogenous murine TSP1 on this phenotype in vitro. Addition of 5 nM TSP1 prevented the loss of ZO-1 from BMEC tight junctions following Krit1 inactivation (FIG. 3a). TSP1 is a modular protein and a recombinant fragment containing 3 Type I repeats (3TSR) (FIG. 11a-11d) that account for much of the anti-angiogenic activity through its capacity to engage both CD36 and integrins^{20,24}. Treatment of BMEC with 20 nM recombinant 3TSR also prevented loss of ZO-1 from cell-cell junctions (FIG. 3b) and the reduction in ZO-1 protein abundance that followed deletion of Krit1 (FIG. 3c, 3d). These effects of 3TSR were also seen in vivo. Treatment of Krit1^{ECKO} mice with 1.6 mg/Kg 3TSR on two succeeding days (FIGS. 12a-12g) increased ZO-1 expression in the cerebellum (FIG. 3e). These data show that replacement of TSP1 can prevent the effects of Krit1 deletion on the distribution and abundance of ZO-1 in brain endothelium and that an anti-angiogenic domain of TSP1 (3TSR) is sufficient for this activity.

TSP1 Replacement with 3TSR Antagonizes Increased VEGFR2 Phosphorylation that Follows Inactivation of Krit1.

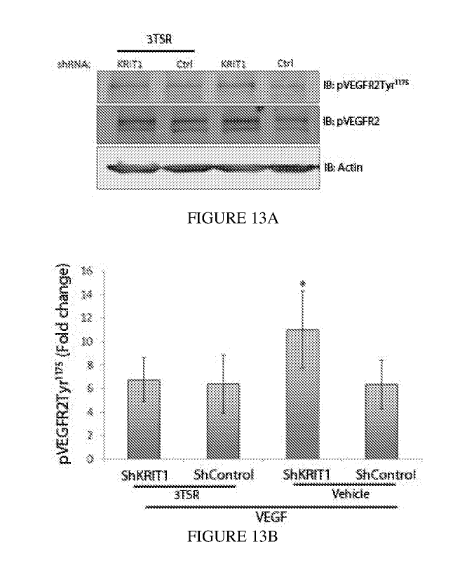

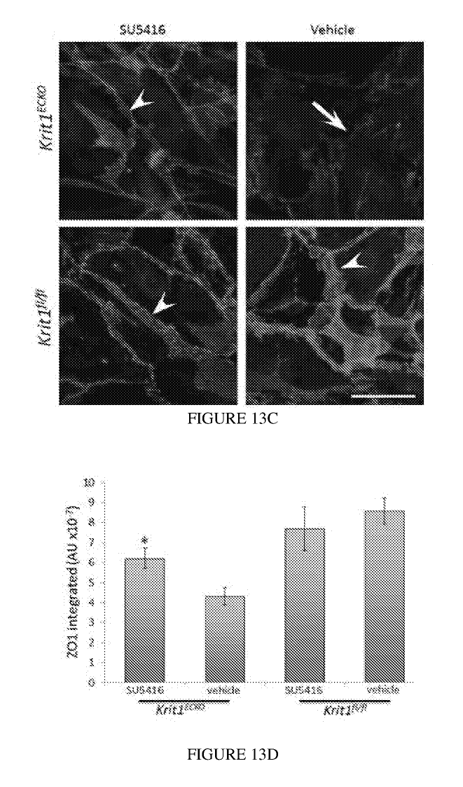

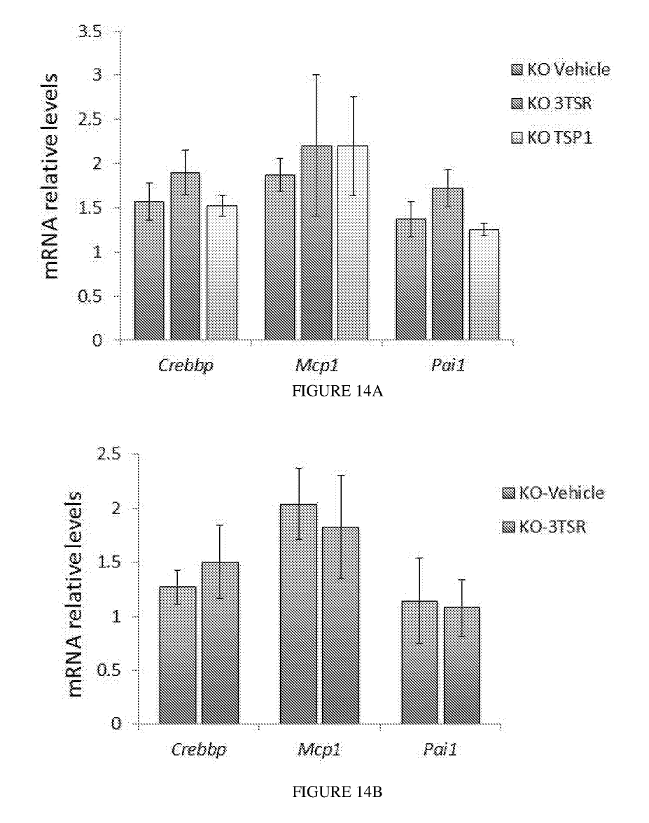

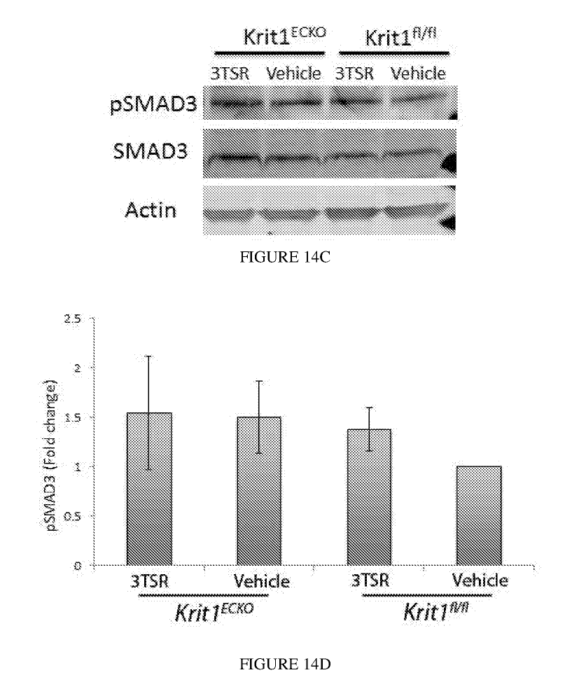

VEGF signaling is enhanced in KRIT1-depleted endothelial cells¹⁴ and can contribute to the disruption of inter-endothelial junctions^{25,26} and capillary dilatation that occur in CCMs²⁷. Therefore, this research assessed the effect of loss of brain endothelial KRIT1 on VEGFR2 phosphorylation as an indicator of VEGFR2 signaling. Immunocytochemistry revealed elevated levels of VEGFR2-Tyr¹¹⁷⁵ phosphorylation in Krit1^{ECKO} BMEC (FIGS. 3f, 3g). Treatment of BMEC with 3TSR prevented the increased VEGFR2-Tyr¹¹⁷⁵ phosphorylation that followed Krit1 deletion (FIGS. 3f, 3g). Furthermore, silencing KRIT1 in human endothelial cells increased VEGFR2 phosphorylation and this was prevented by 3TSR (FIGS. 13a-13d) and a VEGFR2 antagonist ameliorated the effects of KRIT1 deletion on tight junctions (FIGS. 13a-13d). 3TSR also prevented increased VEGFR2-Tyr¹¹⁷⁵ phosphorylation in the brains of Krit1^{ECKO} mice (FIG. 3h). 3TSR can also promote TGF-β activation; however, this research noted no effect of 3TSR on expression of TGF-β-regulated genes or in SMAD3 phosphorylation in Krit1^{ECKO} BMEC (FIGS. 14a-14d). Thus, 3TSR limits the increased VEGFR2 signaling that follows loss of endothelial KRIT1, an effect that can account for both stabilization of tight junctions and prevention of capillary dilation in CCM.

TSP1 Replacement with 3TSR Prevents CCMs.

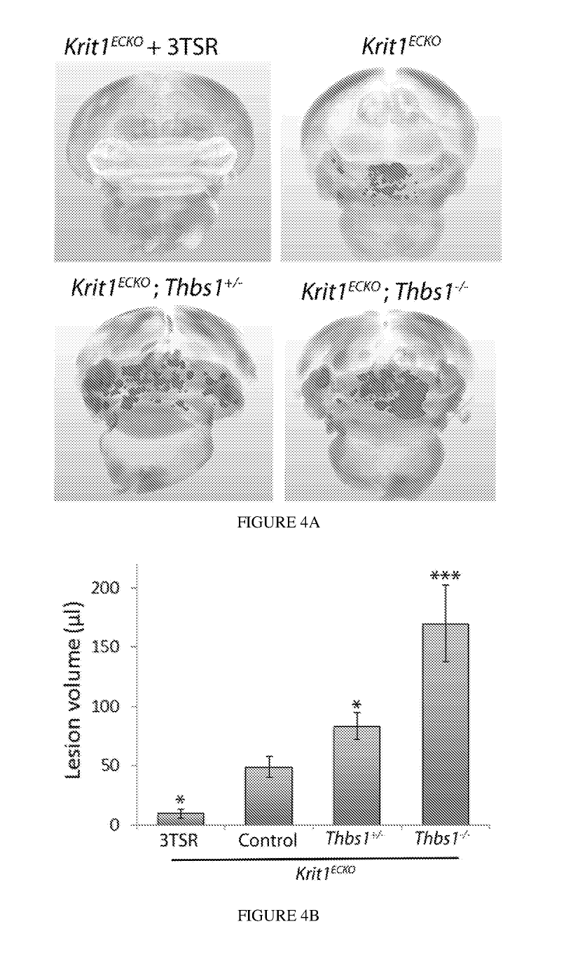

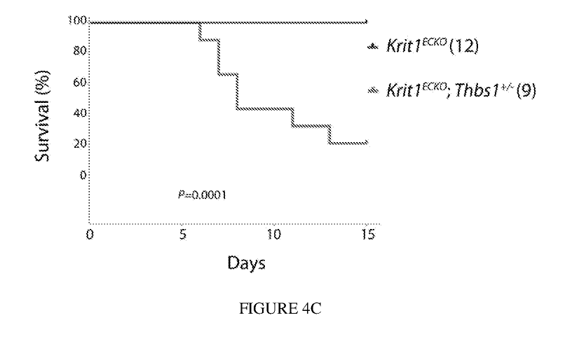

Visual inspection of the hindbrains of neonatal 3TSR-treated Krit1^{ECKO} mice compared with vehicle-treated littermate Krit1^{ECKO} controls revealed a notable reduction in the number and size of vascular lesions (FIGS. 12a-12g). A similar marked reduction in histologically typical CCMs was observed in the 3TSR-treated Krit1^{ECKO} mice (FIGS. 12a-12g). To quantify CCM formation, this example imaged P7 hindbrains using contrast-enhanced, high resolution X-ray micro-computed tomography (microCT), and measured lesion volumes using semi-automated software. 3TSR-treated Krit1^{ECKO} mouse hindbrains exhibited near complete prevention of CCM compared with vehicle-treated Krit1^{ECKO} littermates, as assessed by hindbrain microCT imaging (FIG. 4a). Blinded measurement of total CCM lesion volume (FIG. 4b), confirmed the dramatic reduction in CCM as a consequence of 3TSR administration. The intravenously administered 3TSR was observed in CCM lesions indicating that it can act directly on endothelial cells (FIGS. 12a-12g). Similarly, blinded examination of retinas showed that the extent of condensed peripheral vascular plexus observed at P7 in vehicle-treated Krit1^{ECKO} mice was markedly reduced in Krit1^{ECKO} 3TSR-treated mice (FIGS. 12a-12g). Thus, replacement of TSP1 with 3TSR inhibited CCM formation.

To assess the role of endogenous TSP1 in limiting CCM pathogenesis, this research examined the impact of genetic inactivation of Thbs1 in Krit1^{ECKO} mice. MicroCT analysis of Krit1^{ECKO}; Thbs1^{+/-} mice revealed an ˜80% increase in the volume of CCM lesions relative to Krit1^{ECKO}; Thbs1^{+/+} littermates whereas the surviving Krit1^{ECKO}; Thbs1^{-/-} mice exhibited a ˜200% increase in CCM lesion volume (FIGS. 4a, 4b). Furthermore, there was statistically significant reduction in survival of Krit1^{ECKO}; Thbs1^{+/-} versus Krit1^{ECKO}; Thbs1^{+/+} mice (FIG. 4c). These data show that TSP1 limits the formation of CCMs and that replacement of the loss of TSP1 with an anti-angiogenic fragment can prevent CCMs. Thus, the reduced expression of endothelial TSP1 that follows Krit1 inactivation contributes to CCM lesion pathogenesis.

KLF2 and KLF4 Regulate Expression of TSP1.

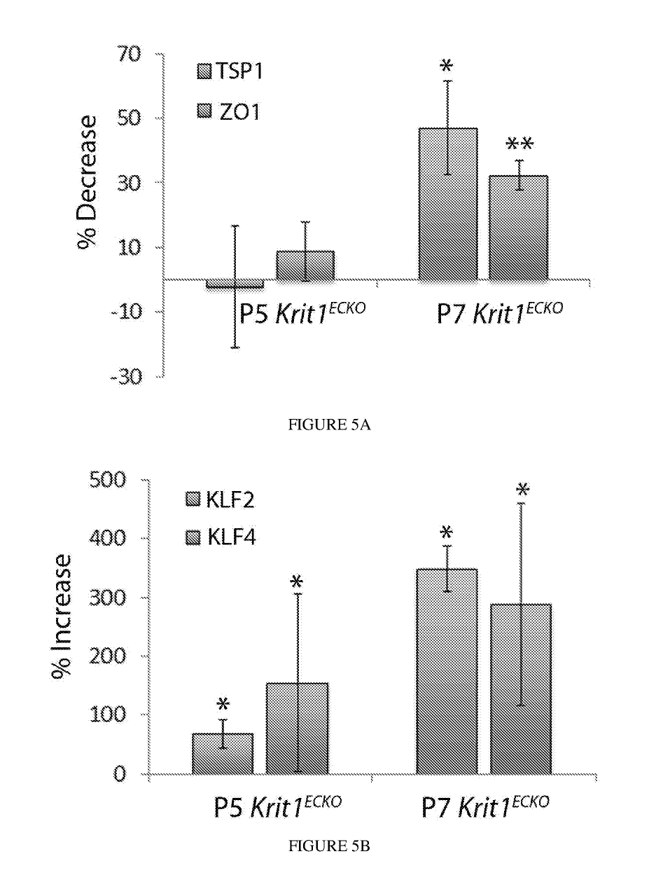

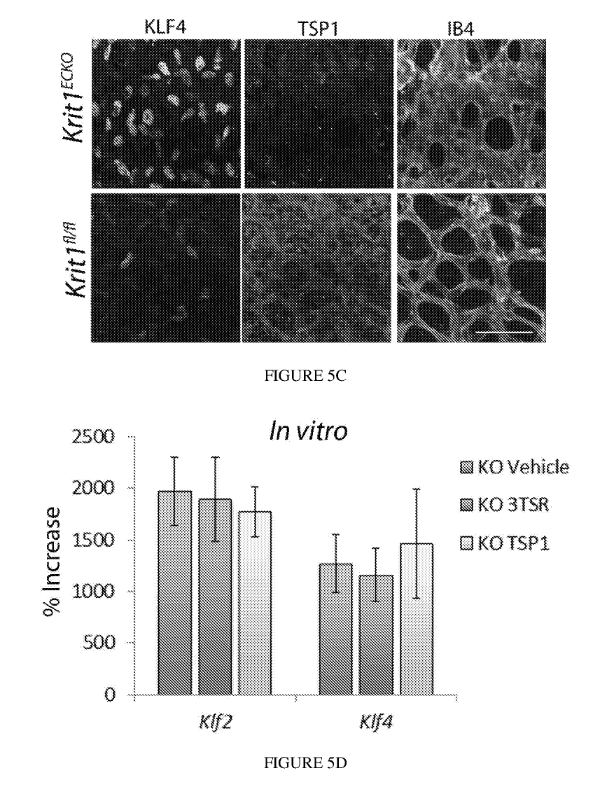

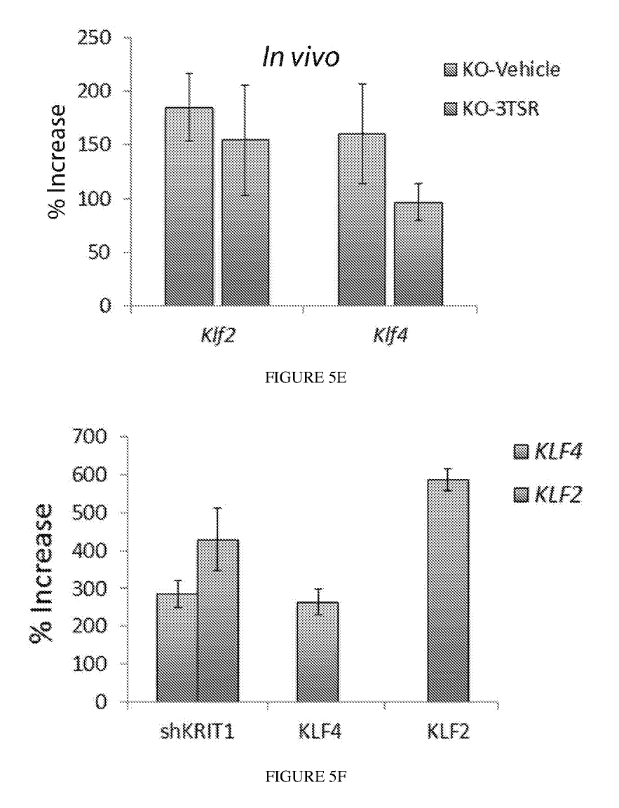

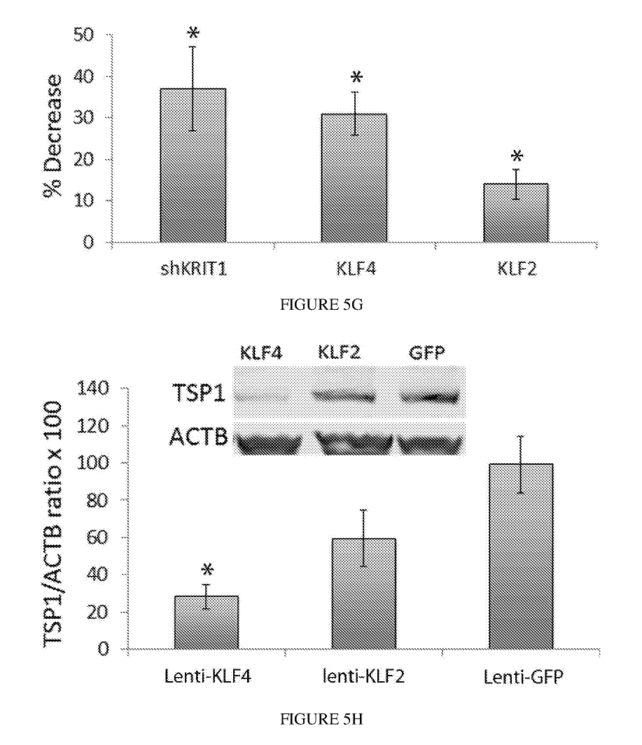

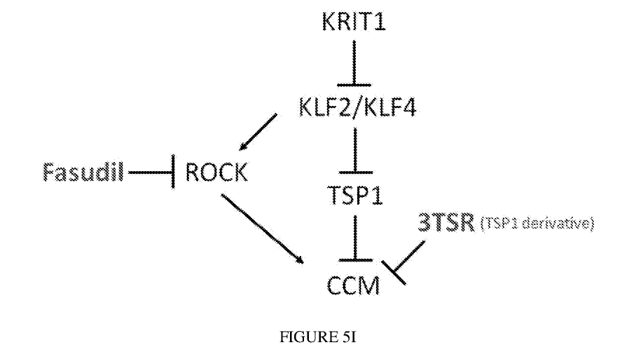

Recent studies established the importance of elevated expression of KLF2 and KLF4 transcription factors in the cardiovascular effects of loss of KRIT1 expression^{7,8,11,12,28} Furthermore, elevation of KLF2 and KLF4 expression precedes an increase in Wnt-3-catenin signaling or an endothelial-mesenchymal transition²⁸. This research noted that both KLF2 and KLF4 expression were increased in freshly-isolated brain microvasculature of Krit1^{ECKO} mice at a time that roughly coincided with the decrease in TSP1 and ZO-1 mRNA levels (FIGS. 5a, 5b). Moreover, in retinas there was marked upregulation of nuclear KLF4 at areas of condensed peripheral vascular plexus⁷ that showed pronounced reduction in TSP1 immunostaining at P7 (FIG. 5c). Because KLF2 and KLF4 are central transcriptional drivers of flow-mediated athero- and thrombo-protective vascular responses^{29,30} and loss of these transcription factors in endothelial cells is lethal in adults³¹, this research examined the effect of TSP1 and 3TSR on expression of these transcription factors. Neither TSP1 nor 3TSR prevented the rise in KLF2 or KLF4 mRNA following Krit1 inactivation in vitro (FIG. 5d) and 3TSR did not do so in vivo (FIG. 5e). Thus, exogenous addition of TSP1 or its active domain, 3TSR, can block vascular effects that follow loss of KRIT1 in spite of maintained elevation of KLF2 and KLF4 expression.

To test whether increased KLF2 and/or KLF4 were sufficient for suppression of TSP1 expression, this research used lentivirus-mediated transduction to ectopically express KLF2 and KLF4 in human endothelial cells at similar levels to those that followed KRIT1 silencing (FIG. 5f). Over expression of KLF2 or KLF4 resulted in ˜15% or ˜30% decrease in TSP1 mRNA, respectively (FIG. 5g). Moreover, ectopic expression of KLF4 induced ˜3.5 fold decrease in TSP1 protein levels in human endothelial cells (FIG. 5h). Whereas the KLF2-induced ˜1.5 fold decrease TSP1 protein abundance was not statistically significant (FIG. 5h). Taken together, these data suggest that the suppression of TSP1 expression is an important downstream effect of the elevation in KLF2 and KLF4 that follows loss of KRIT1 (FIG. 5i). Loss of the angiogenic checkpoint protein, TSP1, then leads to enhanced VEGFR2 signaling that contributes to the pathogenesis of CCMs.

This Example provides that acute Krit1 inactivation in brain microvascular EC (BMEC) causes rapid changes in expression of genes involved in cardiovascular development. Most notable is the dramatic suppression of TSP1, a potent endogenous angiogenesis inhibitor; this suppression is also seen in human CCMs and follows the increase in expression of transcription factors KLF2 and KLF4. Replenishing TSP1 with either full length TSP1 or 3TSR, an anti-angiogenic TSP1 fragment, prevents the disruption of BMEC cell tight junctions, an early phenotypic consequence of loss of KRIT1. Rescue of tight junctions is ascribable to the capacity of 3TSR to prevent increased VEGFR2 phosphorylation in Krit1^{ECKO} BMEC and mice. Administration of 3TSR prevented the development of CCMs in Krit1^{ECKO} mice as judged histologically and by quantitative micro-computerized tomography. Conversely, reduced Thbs1 gene dosage in Krit1^{ECKO} mice increased the CCM lesion burden, demonstrating that endogenous TSP1 limits the pathogenesis of CCM. These studies reveal a critical mechanism in the pathogenesis of CCM and point to the possibility of repurposing 3TSR, a relatively non-toxic angiogenesis inhibitor, for TSP1 replacement therapy of CCM.

Inactivation of brain microvascular endothelial Krit1 induced a rapid change in expression of genes that regulate cardiovascular development. This Example used primary BMECs and a conditional Cre recombinase to precisely control the time of deletion and analyzed gene expression at a time point when KRIT1 mRNA had just fallen to >90% of initial levels. These data provide the first genome-wide view of the acute effects of loss of KRIT1 in the target cell for CCM formation. The dramatic changes in genes tied to the cell cycle and extracellular matrix provides a molecular signature that explains the observed increased proliferation and extracellular matrix seen in lesions from CCM patients³². This Example noted dramatic upregulation of KLF2 and KLF4, transcription factors recently implicated in development of CCM lesions^{7,8,12,28}. Furthermore, confirming recent findings²⁸, little early change was found in genes involved in endothelial-mesenchymal transition, a result explained by Dejana's group's recent report that these markers are elevated ˜15 days after Krit1 inactivation, downstream of the elevation of KLF4⁸. This Example also found several KRIT1-regulated genes encoding secreted proteins and receptors that modulate angiogenic remodeling (e.g., TSP1, CXCR4, BMP2, TGFb2, LRG1, D1)^{6,7,10,16} and inflammation (e.g., TSP1, LBP, ADAM8, NOD2)³². Thus, this analysis provides new molecular clues into the pathogenesis of CCM.

A striking finding was a ˜75% reduction of TSP1 mRNA and ˜70% reduction in TSP1 protein. TSP1 is among the most potent and best-characterized endogenous inhibitors of angiogenesis. Upregulation of expression of TSP1 during angiogenesis limits vascular density, thus serving as an angiogenic checkpoint to prevent neovascularization^{33,34}. Strikingly, in TSP1-null mice, angiogenic vessels are dramatically dilated within tumors^{34,35}, thereby resembling early CCMs. Although loss of KRIT1 can destabilize adherens junctions^{7,36}, this Example found that loss of tight junctions occurs prior to loss of adherens junctions thus mirroring the striking alterations in tight junctions in human CCM lesions^{37,38}. Replacement of TSP1 could prevent the loss of brain endothelial tight junctions that follows inactivation of Krit1 in vitro, indicating that disabling the TSP1 angiogenic checkpoint has a pathogenic role in the increased vascular permeability that characterizes CCMs^{2,5}. Indeed, loss of TSP1 from brain endothelium alters VEGF signaling^{20,34,39}, which can contribute to disassembly of brain endothelial tight junctions²5′²⁶ and cerebrovascular dysfunction^{6,25}. The finding that loss of brain endothelial KRIT1 led to increased levels of VEGFR2 phosphorylation and that VEGF inhibition preserves morphological tight junctions in Krit1^{ECKO} mice are consistent with the report that loss KRIT1 results increases VEGF signaling, and that VEGFR2 inhibitors prevent the resulting increase in endothelial paracellular permeability¹⁴. Thus, 3TSR prevention of increased VEGFR2 signaling in KRIT1^{ECKO} endothelial cells provides a cogent explanation for the capacity of 3TSR to reduce vascular dysmorphology in KRIT1^{ECKO} mice.

It was previously shown that loss of KRIT1 leads to activation of Rho Kinase (ROCK) thereby increasing vascular leak¹⁵ and that blocking ROCK, with inhibitors that are well tolerated in humans, could ameliorate CCMs⁴⁰. Recent data suggest that ROCK activation during CCM formation is downstream of KLF2 elevation²⁸. As shown here, KLF2 and KLF4 suppress TSP1 expression and 3TSR²⁰, which was relatively non-toxic in preclinical studies⁴¹, prevents CCMs without suppressing KLF2 and KLF4. Importantly, 3TSR lacks the TSPI EGF repeats that can disrupt cell-cell junctions⁴². Loss of endothelial KLF2 and KLF4 are lethal³¹ as is inactivation of MEKK3⁴²⁻⁴⁴, which is upstream of the elevation of KLF2²⁸, Thus, this Example indicates that identification of the downstream targets of KLF2 and KLF4 that mediate CCM formation, such as ROCK activation and TSP1 suppression, can serve as a general strategy for discovery of agents that might act alone or synergistically to prevent these common vascular malformations (FIG. 51I).

Genetically-Modified Mice.

The endothelial-specific conditional Krit1 null mice were generated by breeding transgenic mice expressing endothelial specific Pdgfb promoter driven tamoxifen-regulated Cre recombinase, iCreERT2¹⁸, in combination with loxP-flanked Krit1 exon 5 (Krit1^{fl/fl} a generous gift Douglas A. Marchuk, Duke University) (Pdgfb-iCreERT2; Krit1^{fl/fl} mice). All experiments were performed using aged matched Krit1^{fl/fl} littermates on the same C57BL/6 background. TSP1-null (Thbs1^{tm1Hyn}/J, Jackson laboratory) mice were crossed with Pdgfb-iCreERT2; Krit1^{fl/fl} mice to generate Pdgfb-iCreERT2; Krit1^{fl/fl}; TSP1^{+/-} mice. Mice were administered 50 μg of tamoxifen (Sigma, T5648) by intragastric injection on Postnatal days 1, 2 and 3 inducing Cre activity and endothelial Krit1 gene inactivation in the littermates bearing the iCreERT2 (Krit1^{ECKO}). These mice and control Krit1^{fl/fl} mice were sacrificed by decapitation for phenotypic analysis on the indicated postnatal days.

For 3TSR treatment, mice were randomized to TSR (1.6 mg/Kg) or vehicle treatment. Ten μl of 3TSR, were administered by retro-orbital injections on Postnatal day 5 and 6 using a 28-gauge, 0.36 mm×13 mm needle mounted on an insulin syringe (BD Ultra-Fine II, Beckton Dickinson). 3TSR was prepared as described previously⁴⁶. All animal experiments were carried out in compliance with animal procedure protocols approved by the University of California, San Diego Institutional Animal Care and Use Committee.

Isolation and Purification of Primary Brain Microvascular Endothelial Cells (BMEC).

Adult 2 to 4 month old mice were sacrificed and their brains were removed and dropped into ice-cold buffer A (10 mM HEPES, 1× penicillin-streptomycin, 0.5% bovine serum albumin (BSA) in DEMEM). Meninges and choroid plexus were removed, and brain tissue was minced with scissors. Tissue suspension were centrifuged at 700 g for 5 min at 4° C. and the brain tissue pellet was digested with collagenase and dispase solution (DMEM containing 1 mg/ml collagenase/dispase (Sigma, 10269638001), units/ml DNase I (deoxyribonuclear-5′oligonucleotideo-hydrolase, Sigma, 11284932001), 0.150 TLCK (tosyl-lysine-chloromethyl-ketone, Sigma, T7254) at 37° C. for 1 h. After incubation, the brain tissue was triturated with Pasteur pipettes using different size of tips until a homogeneous suspension was obtained. This suspension was centrifuged (700 g for 5 min at 4° C.) and the pellet resuspended in ice-cold buffer B (10 mM HEPES, 1× penicillin streptomycin, 25% BSA in DMEM). The suspension was centrifuged at 1000 g for 20 min at 4° C. The pellet containing microvascular fragments (heavier phase) underwent a second collagenase and dispase digestion for 30 min and was clarified by passage through a 70 m mesh filter. Brain microvascular endothelial cells (BMEC) were seeded onto collagen coated plates, and cultured in EBM-2 medium and supplemented with complements (Lonza, hereafter referred to as EGM-2-BMEC medium) obtained from the manufacturer at the following concentrations: 0.025% (v/v) rhEGF, 0.1% (v/v) IGF, 0.1% (v/v) gentamicin, 0.04% (v/v) ascorbic acid, 0.04% (v/v) hydrocortisone, and 20% (v/v) fetal bovine serum (FBS). BMEC were cultured for two days in 10 μg/ml puromycin and maintained in 2 μg/ml puromycin for 6 days. Purified primary BMEC were routinely characterized for morphology and formation of adherens and tight junctions by immunofluorescence and for the presence of mRNA from endothelial cell-specific genes and absence of leukocyte, glia, and smooth muscle cell marker mRNAs (FIGS. 6a-6e).

Isolation of Brain Microvasculature.

Postnatal day 5-10 mice were euthanized. Brain and cerebellum were removed into ice-cold buffer A. 4 to 5 brains from tamoxifen-injected Krit1^{ECKO} were pooled and comparisons were performed with similar pools from littermate tamoxifen-injected Krit1^{fl/fl} mice. Brain tissue was minced with a scissors and centrifuged at 700 g for 5 min at 4° C. The pellet was resuspended and incubated in ACK lysing buffer (Lonza, 10-548E) for 5 min at room temperature to eliminate erythrocytes. The resulting suspension was sedimented by centrifugation (700 g, 5 min.) and the pellet was digested with collagenase and dispase solution for 1 h at 37° C. The suspension was triturated and centrifuged 700 g for 5 min at 4° C., and microvasculature passed through 70 μm mesh filter. Depletion of blood cell contaminants was performed using Dynabeads Untouched Mouse T cell kit (ThermoFisher, 11413D) following the manufacturer's protocol. For brains at Postnatal stage 7 to 10, the tissue suspension was centrifuged at 700 g for 10 min at 4° C., and pellet was resuspended in ice-cold buffer B and centrifuged at 1000 g for 20 min at 4° C. Density-dependent centrifugation in BSA separates capillary fragments (heavier density) from myelin, neurons, astrocyte and other brain resident contaminants (lighter density). Capillary fragment phase was subject to Percoll gradient as previously reported⁴⁷. For brains in Postnatal 5, tissue suspension was centrifuged at 700 g for 10 min at 4° C., and pellet was resuspended in 100 μl of isolation buffer (Phosphate-buffered saline (PBS), pH 7.2, 0.5% bovine serum albumin (BSA), and 2 mM EDTA) containing microbeads Rat polyclonal antibodies anti-mouse CD31 (Miltenyi Biotec, 130-097-418). After incubation, cell suspension was washed using 7 ml isolation buffer and centrifuged at 1000 g for 10 min. Supernatant was removed and cell pellet resuspended in 500 μl of isolation buffer. CD31+ cells were sorted by applying cell suspension onto LS column placed into a magnetic field following manufacturer's protocol (Miltenyi Biotec, 130-042-401). Endothelial cell identity was confirmed by RT-qPCR of mRNA from endothelial cell-specific genes and minor levels of leukocyte, glia, and smooth muscle cell marker mRNAs (FIGS. 8a-8c).

Genetic Inactivation of KRIT1 in BMEC.

BMEC at passages 1-3 were maintained at 37° C. in 95% air and 5% CO₂ and were grown to 85% confluence and treated for 48 h with 5 μM 4-hydroxy-tamoxifen (Sigma, H7904) after which the medium was replaced with medium lacking 4-hydroxy-tamoxifen and cells were harvested after 72 hr in culture. For TSP1 and 3TSR addition experiments, after 24 h in regular medium, the 5 nM mrTSP1 (R&D systems, 7859-TH), 20 nM 3TSR, or vehicle were added. After 48 hr a second dose of TSP1, 3TSR, or vehicle were added and cells were harvested after an additional 24 h (FIG. 11a-11d).

Immunofluorescence Microscopy.

BMEC were grown to confluence on collagen coated cover glass (Fisher Scientific, 12-545-81) and cells were fixed for 10 min at room temperature with 4% paraformaldehyde (PFA) in phosphate-buffered saline (PBS; pH 7.4) and then permeabilized with 0.5% Triton X-100 in PBS for 5 min. The slides were blocked with 0.5% BSA for 30 min and incubated with Rabbit polyclonal antibodies anti-ZO-1 (1:80; ThermoFisher, 61-7300), Mouse monoclonal antibodies anti-CLDN5 (1:50; ThermoFisher, 35-2500), and Rat polyclonal antibodies anti-VEcadherin (1:100; BD Pharmigen, 550548) overnight at room temperature. For VEGF receptor 2 (VEGFR2) phosphorylation, BMEC were grown to subconfluence on collagen coated cover glass and fixed in methanol for 30 min at 4° C., followed by cold acetone (maintained at −20° C. before use) for 1 min at room temperature and incubated with anti-pVEGFR2Tyr¹¹⁷⁵ antibody (1:100; Cell signaling). Cells were washed 4 times in PBS and incubated for 1 h at room temperature (RT) with a suitable Alexa-Fluor coupled secondary antibody (1:300, ThermosFisher) in PBS. Cell nuclei were stained with (4′,6-diamidino-2-phynylindole) DAPI and mounted with Fluoromount-G mounting media (SouthernBiotech).

RNA Extraction and Quantitative RT-PCR.

Primary BMEC, human umbilical vein endothelial cells (HUVEC) and freshly isolated brain microvasculature total RNA were isolated using Trizol reagent, according to the manufacturer's protocol (ThermoFisher). For gene expression analysis, single stranded cDNA was produced from 10 ng of total RNA of BMEC using SuperScript III First-Strand synthesis and random primers according to the manufacture's protocol (ThermoFisher). KAPPA SyberFast qPCR kit (Kapa Biosystems) and thermal cycler (CFX96 Real-Time System, Bio-Rad) were used to determine the relative levels of the genes analyzed (primer sequences not shown) according to the manufacturer's protocol. Actin mRNA levels were used as an internal control and the 2^{-ΔΔCT} method was used for analysis of the data. Each control value (Krit1^{fl/fl}) was normalized to one, and Krit1^{ECKO} values were relative to control.

Genome-Wide RNA Sequencing.

The quantity (1000 spectrophotometer; Nano-Drop Technologies) and the quality (Agilent Tapestation) of the total RNA were analyzed. RNA libraries were generated using Illumina's TruSeq Stranded mRNA Sample Prep Kit using 400 ng of RNA. RNA libraries were multiplexed and sequenced with 100 base pair (bp) paired single end reads (SR100) to a depth of approximately 30 million reads per sample on an Illumina HiSeq2500. Fastq files from RNA-seq experiments were mapped to individual genome for the mouse strain of origin using STAR with default parameters (mm10 for C57BL/6J)⁴⁸. Reproducibility between samples was analyzed using Irreproducibility discovery rate tool (IDR)⁴⁹. Homer⁵⁰ was used for further analysis. To measure gene expression, analyze repeats with the option RNA and condense Genes along with the default parameters was used. Subset-specific expression was defined with a 4-fold difference in expression between two experiments. Genes with less than 16 tag counts were defined as not expressed. To map subset-specific peaks to gene expression, only expressed genes were considered. Differential expression was defined by a fold-change of at least 1.5-fold averaging over replicates (P<0.05) and used for GO annotation analysis with DAVID Bioinformatics^{45,51} Sequencing data have been deposited in Gene Expression Omnibus (GEO) under accession number GSE85657.

Whole-Mount Retinal Staining.

Eyes were harvested and fixed in PFA for 20 min at RT. Eyes were washed 4 times with PBS, and retinal whole-mount preparation was permeabilized and blocked using blocking buffer (PBS, 1% BSA and 0.5% Triton X-100) and incubating at 4° C. overnight for tight junction and two days for KLF4 staining. For Tight junctions and adherens junction staining, whole-mount preparations were incubated with rabbit polyclonal antibodies anti-ZO1 (1:50), mouse monoclonal antibody anti-CLDN5 (1:80) and Rat polyclonal antibodies anti-VE-cadherin (1:100) in PBS for overnight at RT. For KLF4 and TSP1 staining, whole-mount preparations were incubated with Goat polyclonal antibodies anti-KLF4 (1:100, R&D systems, AF3158) and Rabbit polyclonal antibodies anti-TSP1 (1:500) in PBS at RT for one day follow by 4° C. for two days. Retinal whole-mount preparation was washed 3 times in PBS and 3 times in Pblec buffer (PBS, 1 mM CaCl2, 1 mM MgCl2, 0.1 m M MnC12 and 1% Triton X-100) and incubated with isolectin B₄ FITC (1:80, Sigma, L2895) or Alexa-647 (1:80, ThermosFisher, 132450) conjugated, as indicated, in Pblec buffer (1 mM CaCl2, 1 mm MgCl2, 0.1 mM MnC12, 0.1% Triton X100 in PBS) at 4° C. overnight. Retinal whole-mount preparations were incubated at RT for 2 h with a suitable secondary anti-rabbit Alexa 594, anti-goat Alexa 488 and anti-mouse Alexa 647 antibodies (1:250, ThermoFisher) in PBS. Retinal whole-mount preparations were washed 5 times in PBS and flat-mounted using Fluoromount-G (SouthernBiotech).

Immunohistochemistry.

Postnatal day 7 Krit1^{ECKO} and littermate control Krit1^{fl/fl} mice were perfused with Hank's balanced salt solution containing 0.5% BSA, to rinse out the blood, and brains were isolated and fixed in PFA 4% at 4° C. overnight. After cryoprotection in sucrose and freezing, 12 m sections of cerebellar tissue were cut onto Superfrost Plus slides (VWR international, 12-550-15). The preparation was blocked and permeabilized using permeabilization buffer (PBS, 5% goat serum, 0.5% triton X-100 and 0.5% BSA) for 2 h and incubated with Rabbit polyclonal antibodies anti-TSP1 (1:1000), Rat polyclonal antibodies anti-PECAM1 (1:100, BD Pharmingen, 553370), Rabbit polyclonal antibodies anti-ZO1 (1:120), Mouse monoclonal antibody anti-CLDN5 (1:100), or Rat polyclonal antibodies anti-VE-cadherin (1:100) in PBS. All antibodies were incubated at RT overnight in a humidified box. For human tissue, CCM1 lesions and lesion-free brain tissues were snap frozen and sectioning using cryostat (Leica). Specimens were fixed in PFA 4% at room temperature for 15 min, and washed three times using PBS. The specimens were blocked and permeabilized using permeabilization buffer for 3 h and incubated with Rabbit polyclonal antibodies anti-TSP1 (1:1000), Goat polyclonal antibodies anti-collagen IV (1:100, Millipore, AB769) in PBS at room temperature overnight. Preparations were washed 4 times in PBS and incubated at RT for 1 h with a suitable secondary anti-rabbit Alexa 594, anti-Goat Alexa 488 or anti-Rat Alexa 488, and anti-mouse Alexa 647 antibodies (1:300, Thermoscientific) in PBS. Cell nuclei were stained with DAPI (SouthernBiotech).

Image Acquisition and Quantitative Analysis.

The slides were viewed with a high-resolution SP5 confocal microscope (Leica Microsystems) and the images were captured with Leica application suite (LAS) software (Leica Microsystems). For BMEC confocal microscopy, 5 images in Z stacks up to 4.5 μm in depth were acquired with a X60 oil-immersion objective and projected onto 1 image. For cerebellar tissue and retinas confocal microscopy, 6 images in Z stacks up to 8 m (brain) or 6 m (retinas) in depth were acquired with a X20 objective or 6 images to 6 μm in depth were acquired with a X60 oil-immersion objective and projected onto 1 image. The quantification analysis was performed using Volocity® software on high-resolution confocal images.

Western Blot Analysis.

BMEC or HUVEC as indicated were grown to confluence on collagen-coated 6-well plates whereas cerebellar frozen tissue was immersed in liquid nitrogen and pulverized by tissuecrusher. Cells and tissue were lysed using lysis solution containing 100 l RIPA buffer (25 mM Tris-HCl pH 7.6, 150 mM NaCl, 1% Nonidet P-40, 1% sodium deoxycholate, 0.1% SDS) and a mixture of inhibitors (Roche) and 1 mM sodium orthovanadate. Protein concentration was determined using a Micro BCA protein assay kit (Pierce). Cell lysates were diluted in Laemmli's buffer solution at 95° C. for 5 min. Twenty-five micrograms of total protein was resolved on 4-20% polyacrylamide gels (ThermoFisher) in SDS-PAGE buffer and transferred onto nitrocellulose membranes (Amersham) using a wet transfer system (Bio-Rad). Membranes were blocked with blocking WB solution (PBS, 10% nonfat milk and 0.05% Tween-20) for 1 h and incubated in the presence of Rabbit polyclonal antibodies anti-TSP1 (1:500, Abcam, ab85762) anti-ZO-1 (1:150), Rat polyclonal antibodies anti-VE-cadherin (1:75), or Rabbit polyclonal antibodies anti-claudin-5 (1:170, ThermoFisher, 34-1600), Rabbit polyclonal anti-VEGFR2 (1:200 Cell signaling and 1:200 Santa Cruz), Rabbit monoclonal anti-VEGFR2pTyr¹¹⁷⁵ (1:120 Cell Signaling) at 4° C. overnight. After several washes, membranes were incubated with the appropriate IRDye/Alexa-Fluor coupled secondary antibody (1:10,000, Li-Cor) and imaged using an infrared imaging system (Odyssey; Li-Cor). Blots were processed using Image Studio Lite software (Li-Cor). As a control for protein loading mouse antibodies against actin (1:5000) (Sigma-Aldrich, A1978) were used.

Micro-CT Scan Image.

Postnatal day 7 Krit1^{ECKO}, Krit1^{ECKO}; TSP1^{+/-}, Krit1^{ECKO}; TSP1^{-/-} and littermate control Krit1^{fl/fl}, Krit1^{+/-}; TSP1^{+/-,} Krit1^{fl/fl}; TSP1^{-/-} mice were euthanized and their brains were removed and dropped into 10% neutral buffered formalin (Sigma-Aldrich, St. Louis, Mo., USA). The brains were soaked in 50 ml of 1.25% Lugol's iodine (Thermo Fisher Scientific, Waltham, Mass., USA) during 96 hours. The imaging data acquisition was performed using the Phoenix vltomelx s 180/240 micro-CT scanner system (General Electric, Fairfield, Conn., USA) and the hyper-dense CCM lesions were computationally segmented AMIRA 5.5 software platform (FEI, Hillsboro, Oreg., USA)⁵².

Expression Constructs and ShRNAs.

To generate the KLF2 and KLF4 constructs, a plasmid template encoding KLF2 (Addgene Cat No. 50786) and KLF4 (Addgene Cat No. 19764) were amplified by PCR to place them downstream of mouse PGK promoter and fused to IRES-puromycin resistance gene into pLVX vector (Clonetech). KLF2- and KLF4-expressing lentiviral particles were prepared by co-transfection of pLVX; PGK-KLF2 or pLVX; PGK-KLF4 with pMDLg/pRRE, pRSV-Rev, and pMD2.G in HEK293T cells. Oligos for ShKRIT1 (clone TRCN0000072879) is based on the public TRC (The RNAi consortium, Broad Institute) library and cloned into pLKO.1 using EcoRI and Age1 restriction sites. For KLF2, KLF4, or ShRNA delivery, HUVEC were grown to 80% confluence on gelatin coated 6 well plate, and then transduced with lentiviral particles. 72 hours post-infection, HUVEC were prepared for RNA or protein analysis (described above).

Statistical Analysis.

Data are expressed as means+/−standard error of the mean (S.E.M). For all experiments, the number of independent experiments (N) is indicated. Analyses of brain and retina experiments were performed blinded. The sample sizes were estimated with two-sample t test (two tailed). The pooled standard deviation was used to account for the unequal variances in the two groups (Vehicle or 3TSR). Two-tailed unpaired Student's t-test was used to determine statistical significance. For multiple comparisons, one-way ANOVA follow by Tukey's post hoc test was used. For survival rate analysis, long-rank test was performed using Software SAS (*P<0.05, **P<0.01, ***P<0.001).

### REFERENCES

1. Golden, M. J., Morrison, L. A., Kim, H. & Hart, B. L. Increased number of white matter lesions in patients with familial cerebral cavernous malformations. AJNR. American journal of neuroradiology 36, 899-903, doi:10.3174/ajnr.A4200 (2015).
2. Mikati, A. G. et al. Vascular permeability in cerebral cavernous malformations. Journal of cerebral blood flow and metabolism: official journal of the International Society of Cerebral Blood Flow and Metabolism, doi:10.1038/jcbfm.2015.98 (2015).
3. McDonald, D. A. et al. Lesions from patients with sporadic cerebral cavernous malformations harbor somatic mutations in the CCM genes: evidence for a common biochemical pathway for CCM pathogenesis. Human molecular genetics 23, 4357-4370, doi:10.1093/hmg/ddul53 (2014).
4. D'Angelo, R. et al. Mutation analysis of CCM1, CCM2 and CCM3 genes in a cohort of Italian patients with cerebral cavernous malformation. Brain pathology 21, 215-224, doi:10.1111/j.1750-3639.2010.00441.x (2011).
5. Tanriover, G. et al. Ultrastructural analysis of vascular features in cerebral cavernous malformations. Clinical neurology and neurosurgery 115, 438-444, doi:10.1016/j.clineuro.2012.06.023 (2013).
6. Boulday, G. et al. Developmental timing of CCM2 loss influences cerebral cavernous malformations in mice. The Journal of experimental medicine 208, 1835-1847, doi:10.1084/jem.20110571 (2011).
7. Maddaluno, L. et al. EndMT contributes to the onset and progression of cerebral cavernous malformations. Nature 498, 492-496, doi:10.1038/nature12207 (2013).
8. Cuttano, R. et al. KLF4 is a key determinant in the development and progression of cerebral cavernous malformations. EMBO molecular medicine 8, 6-24, doi:10.15252/emmm.201505433 (2015).
9. McDonald, D. A. et al. A novel mouse model of cerebral cavernous malformations based on the two-hit mutation hypothesis recapitulates the human disease. Human molecular genetics 20, 211-222, doi: 10.1093/hmg/ddq433 (2011).
10. Kevin J. Whitehead, N. W. P., Jennifer A. Adams, Douglas A. Marchuk and & Li, D. Y. Ccml is required for arterial morphogenesis: implications for the etiology of human cavernous malformations. Development and disease March; 131(6): 1437-1448 (2003).
11. Zhou, Z. et al. The cerebral cavernous malformation pathway controls cardiac development via regulation of endocardial MEKK3 signaling and KLF expression. Developmental cell 32, 168-180, doi:10.1016/j.devcel.2014.12.009 (2015).
12. Renz, M. et al. Regulation of beta1 integrin-Klf2-mediated angiogenesis by CCM proteins. Developmental cell 32, 181-190, doi:10.1016/j.devcel.2014.12.016 (2015).
13. Wustehube, J. et al. Cerebral cavernous malformation protein CCM1 inhibits sprouting angiogenesis by activating DELTA-NOTCH signaling. Proceedings of the National Academy of Sciences of the United States of America 107, 12640-12645, doi:10.1073/pnas.1000132107 (2010).
14. DiStefano, P. V., Kuebel, J. M., Sarelius, I. H. & Glading, A. J. KRIT1 protein depletion modifies endothelial cell behavior via increased vascular endothelial growth factor (VEGF) signaling. The Journal of biological chemistry 289, 33054-33065, doi:10.1074/jbc.M114.582304 (2014).
15. Stockton, R. A., Shenkar, R., Awad, I. A. & Ginsberg, M. H. Cerebral cavernous malformations proteins inhibit Rho kinase to stabilize vascular integrity. The Journal of experimental medicine 207, 881-896, doi:10.1084/jem.20091258 (2010).
16. Whitehead, K. J. et al. The cerebral cavernous malformation signaling pathway promotes vascular integrity via Rho GTPases. Nature medicine 15, 177-184, doi:10.1038/nm.1911 (2009).
17. Lampugnani, M. G. et al. CCM1 regulates vascular-lumen organization by inducing endothelial polarity. J Cell Sci 123, 1073-1080, doi:123/7/1073 [pii]10.1242/jcs.059329 [doi] (2010).
18. Claxton, S. et al. Efficient, inducible Cre-recombinase activation in vascular endothelium. Genesis (New York, N.Y.: 2000) 46, 74-80, doi:10.1002/dvg.20367 (2008).
19. Zhang, X. & Lawler, J. Thrombospondin-based antiangiogenic therapy. Microvascular research 74, 90-99, doi:10.1016/j.mvr.2007.04.007 (2007).
20. Lawler, P. R. & Lawler, J. Molecular basis for the regulation of angiogenesis by thrombospondin-1 and -2. Cold Spring Harbor perspectives in medicine 2, a006627, doi:10.1101/cshperspect.a006627 (2012).
21. Mikati, A. G. et al. Vascular permeability in cerebral cavernous malformations. Journal of cerebral blood flow and metabolism: official journal of the International Society of Cerebral Blood Flow and Metabolism 35, 1632-1639, doi:10.1038/jcbfm.2015.98 (2015).
22. Glading, A., Han, J., Stockton, R. A. & Ginsberg, M. H. KRIT-1/CCM1 is a Rapl effector that regulates endothelial cell cell junctions. The Journal of cell biology 179, 247-254, doi:10.1083/jcb.200705175 (2007).
23. Pitulescu, M. E., Schmidt, I., Benedito, R. & Adams, R. H. Inducible gene targeting in the neonatal vasculature and analysis of retinal angiogenesis in mice. Nature protocols 5, 1518-1534, doi:10.1038/nprot.2010.113 (2010).
24. Short, S. M. et al. Inhibition of endothelial cell migration by thrombospondin-1 type-1 repeats is mediated by beta1 integrins. The Journal of cell biology 168, 643-653, doi:10.1083/jcb.200407060 (2005).
25. Argaw, A. T. et al. Astrocyte-derived VEGF-A drives blood-brain barrier disruption in CNS inflammatory disease. The Journal of clinical investigation 122, 2454-2468, doi: 10.1172/JCI60842 (2012).
26. Morin-Brureau, M. et al. Epileptiform activity induces vascular remodeling and zonula occludens 1 downregulation in organotypic hippocampal cultures: role of VEGF signaling pathways. The Journal of neuroscience: the official journal of the Society for Neuroscience 31, 10677-10688, doi:10.1523/JNEUROSCI.5692-10.2011 (2011).
27. Jung, K. H. et al. Cerebral cavernous malformations with dynamic and progressive course: correlation study with vascular endothelial growth factor. Archives of neurology 60, 1613-1618, doi:10.1001/archneur.60.11.1613 (2003).
28. Zhou, Z. et al. Cerebral cavernous malformations arise from endothelial gain of MEKK3-KLF2/4 signalling. Nature 532, 122-126, doi:10.1038/naturel7178 (2016).
29. Lin, Z. et al. Kruppel-like factor 2 (KLF2) regulates endothelial thrombotic function. Circulation research 96, e48-57, doi:10.1161/01.RES.0000159707.05637.a1 (2005).
30. Zhou, G. et al. Endothelial Kruppel-like factor 4 protects against atherothrombosis in mice. The Journal of clinical investigation 122, 4727-4731, doi: 10.1172/JCI66056 (2012).
31. Sangwung, P. et al. KLF2 and KLF4 control endothelial identity and vascular integrity. JCI insight 2, e91700, doi:10.1 172/jci.insight.91700 (2017).
32. Shenkar, R., Shi, C., Check, I. J., Lipton, H. L. & Awad, I. A. Concepts and hypotheses: inflammatory hypothesis in the pathogenesis of cerebral cavernous malformations. Neurosurgery 61, 693-702; discussion 702-693, doi:10.1227/01.NEU.0000298897.38979.07 (2007).
33. Jimenez, B. et al. Signals leading to apoptosis-dependent inhibition of neovascularization by thrombospondin-1. Nature medicine 6, 41-48, doi:10.1038/71517 (2000).
34. Rodriguez-Manzaneque, J. C. et al. Thrombospondin-1 suppresses spontaneous tumor growth and inhibits activation of matrix metalloproteinase-9 and mobilization of vascular endothelial growth factor. Proceedings of the National Academy of Sciences of the United States of America 98, 12485-12490, doi:10.1073/pnas.171460498 (2001).
35. Velasco, P. et al. The angiogenesis inhibitor thrombospondin-1 inhibits acute cutaneous hypersensitivity reactions. The Journal of investigative dermatology 129, 2022-2030, doi: 10.1038/jid.2008.447 (2009).
36. Glading, A. J. & Ginsberg, M. H. Rapl and its effector KRIT1/CCM1 regulate beta-catenin signaling. Disease models & mechanisms 3, 73-83, doi:10.1242/dmm.003293 (2010).
37. Stamatovic, S. M., Sladojevic, N., Keep, R. F. & Andjelkovic, A. V. PDCD10 (CCM3) regulates brain endothelial barrier integrity in cerebral cavernous malformation type 3: role of CCM3-ERK1/2-cortactin cross-talk. Acta neuropathologica 130, 731-750, doi:10.1007/s00401-015-1479-z (2015).
38. Schneider, H. et al. Impairment of tight junctions and glucose transport in endothelial cells of human cerebral cavernous malformations. Journal of neuropathology and experimental neurology 70, 417-429, doi:10.1097/NEN.0b013e31821bc40e (2011).
39. Isenberg, J. S., Martin-Manso, G., Maxhimer, J. B. & Roberts, D. D. Regulation of nitric oxide signalling by thrombospondin 1: implications for anti-angiogenic therapies. Nature reviews. Cancer 9, 182-194, doi:10.1038/nrc2561 (2009).
40. McDonald, D. A. et al. Fasudil decreases lesion burden in a murine model of cerebral cavernous malformation disease. Stroke; a journal of cerebral circulation 43, 571-574, doi:10.1161/STROKEAHA.111.625467 (2012).
41. Russell, S. et al. Combined therapy with thrombospondin-1 type I repeats (3TSR) and chemotherapy induces regression and significantly improves survival in a preclinical model of advanced stage epithelial ovarian cancer. FASEB journal: official publication of the Federation of American Societies for Experimental Biology 29, 576-588, doi:10.1096/fj.14-261636 (2015).
42. Hamik, A. et al. Kruppel-like factor 4 regulates endothelial inflammation. The Journal of biological chemistry 282, 13769-13779, doi:10.1074/jbc.M700078200 (2007).
43. Parmar, K. M. et al. Integration of flow-dependent endothelial phenotypes by Kruppel-like factor 2. The Journal of clinical investigation 116, 49-58, doi: 10.1172/JCI24787 (2006).
44. Ohnesorge, N. et al. Erk5 activation elicits a vasoprotective endothelial phenotype via induction of Kruppel-like factor 4 (KLF4). The Journal of biological chemistry 285, 26199-26210, doi:10.1074/jbc.M110.103127 (2010).
45. Huang da, W., Sherman, B. T. & Lempicki, R. A. Systematic and integrative analysis of large gene lists using DAVID bioinformatics resources. Nature protocols 4, 44-57, doi:10.1038/nprot.2008.211 (2009).
46. Miao, W. M. et al. Thrombospondin-1 type 1 repeat recombinant proteins inhibit tumor growth through transforming growth factor-beta-dependent and -independent mechanisms. Cancer research 61, 7830-7839 (2001).
47. Lopez-Ramirez, M. A. et al. MicroRNA-155 negatively affects blood-brain barrier function during neuroinflammation. FASEB journal: official publication of the Federation of American Societies for Experimental Biology 28, 2551-2565, doi:10.1096/fj.13-248880 (2014).
48. Dobin, A. et al. STAR: ultrafast universal RNA-seq aligner. Bioinformatics 29, 15-21, doi: 10.1093/bioinformatics/bts635 (2013).
49. Li, Q. H., Brown, J. B., Huang, H. Y. & Bickel, P. J. Measuring Reproducibility of High-Throughput Experiments. Ann Appl Stat 5, 1752-1779, doi:10.1214/11-AOAS466 (2011).
50. Heinz, S. et al. Simple combinations of lineage-determining transcription factors prime cis-regulatory elements required for macrophage and B cell identities. Molecular cell 38, 576-589, doi: 10.1016/j.molcel.2010.05.004 (2010).
51. Eichenfield, D. Z. et al. Tissue damage drives co-localization of NF-kappaB, Smad3, and Nrf2 to direct Rev-erb sensitive wound repair in mouse macrophages. eLife 5, doi:10.7554/eLife.13024 (2016).
52. Girard, R. et al. Micro-computed tomography in murine models of cerebral cavernous malformations as a paradigm for brain disease. Journal of neuroscience methods 271, 14-24, doi:10.1016/j.jneumeth.2016.06.021 (2016).

1. A method for treating cerebral cavernous malformations, comprising:
administering to a subject in need thereof a pharmaceutical composition comprising a therapeutically effective amount of a thrombospondin 1 protein agent, thereby treating cerebral cavernous malformations in the patient.

2. The method of claim 1, wherein the thrombospondin 1 protein agent is thrombospondin 1 protein.

3. The method of claim 1, wherein the thrombospondin 1 protein agent is a biologically functional fragment of thrombospondin 1 protein.

4. The method of claim 3, wherein the biologically functional fragment of thrombospondin 1 protein is 3TSR.

5. The method of claim 1, wherein the thrombospondin 1 protein agent is a thrombospondin 1 protein isomer.

6. The method of claim 1, wherein the thrombospondin 1 protein agent is a homolog of thrombospondin 1 protein.

7. The method of claim 1, wherein the thrombospondin 1 protein agent is a functional fragment of a homolog of thrombospondin 1 protein.

8. The method of claim 1, wherein the thrombospondin 1 protein agent is a peptidomimetic of thrombospondin 1 protein or a functional fragment thereof.

9. The method of claim 8, wherein the peptidomimetic of thrombospondin 1 protein is ABT-510.

10. The method of claim 1, wherein the subject is a human.

11. The method of claim 1, wherein the subject has a mutated KRIT1 gene.

12. The method of claim 1, wherein the pharmaceutical composition is administered orally.

13. The method of claim 1, wherein the step of administering the pharmaceutical composition reduces the subject's risk of developing vascular lesions.

14. The method of claim 1, wherein the step of administering the pharmaceutical composition prevents the subject from developing vascular lesions.

15. The method of claim 1, wherein the step of administering the pharmaceutical composition reverts the subject's vascular lesions to a non-diseased state.

16. The method of claim 1, further comprising administering to the subject a therapeutically effective amount of a Rho Kinase inhibitor.

17. The method of claim 16, wherein the pharmaceutical composition further comprises the Rho Kinase inhibitor.

18. (canceled)

19. A pharmaceutical composition comprising a pharmaceutically acceptable excipient and a therapeutically effective amount of a thrombospondin 1 protein agent of to treat cerebral cavernous malformations in a patient.

20. The pharmaceutical composition of claim 19 further comprising a Rho Kinase inhibitor.
